(19) United States
(12) Patent Application Publication
(10) Pub. No.: US 2023/0348988 A1
(43) Pub. Date: Nov. 2, 2023

(54) DNA Damage Repair Deficit in Cancer Cells

(71) Applicants: THE REGENTS OF THE UNIVERSITY OF CALIFORNIA, Oakland, CA (US); FUNDACIO INSTITUT DINVESTIGACIO BIOMEDICA DE BELLVITGE, Barcelona (ES); INSTITUT CATALA DONCOLOGIA, Barcelona (ES)

(72) Inventors: Mary Helen Barcellos-Hoff, San Francisco, CA (US); Qi Liu, San Francisco, CA (US); Miquel Angel Pujana, Barcelona (ES)

(21) Appl. No.: 18/009,885

(22) PCT Filed: Jun. 11, 2021
(86) PCT No.: PCT/US21/37078

§ 371 Date: Dec. 12, 2022

Related U.S. Application Data
(60) Provisional application No. 63/038,747, filed on Jun. 12, 2020.

(51) Int. Cl. C12Q 1/6886; A61K 31/282; A61K 31/55; A61K 45/06; A61P 35/00; A61K 51/10

(52) U.S. Cl. CPC C12Q 1/6886; A61K 31/282; A61K 31/55; A61K 45/06; A61P 35/00; A61K 51/1045; C12Q 2600/158

(57) ABSTRACT

Presented are methods of assessing a deficit in DNA damage repair capability in cancer cells. Cancer cells having this DNA damage repair phenotype have increased susceptibility to certain treatments, including genotoxic treatments, treatment with PARP1 inhibitors, and immunotherapies. The methods encompass the use of novel gene expression signatures that enable facile and rapid determination of the DNA damage repair phenotype in cancer cells of a sample. By these methods, subjects having cancer that is amenable to genotoxic treatment, PARP1 inhibition, or immunotherapy may be identified and administered a suitable treatment. Also disclosed is a method of inducing the DNA damage repair deficit phenotype in cancer cells to sensitize them to various treatments.

### CROSS-REFERENCE TO RELATED APPLICATIONS

This application claims the benefit of priority to U.S. Provisional Patent Application Ser. No. 63/038,747 entitled “DNA Damage Repair Deficit in Cancer Cells,” filed Jun. 12, 2020, the contents of which are hereby incorporated by reference.

### STATEMENT REGARDING FEDERALLY SPONSORED RESEARCH OR DEVELOPMENT

Not Applicable.

### BACKGROUND OF THE INVENTION

The cytokine transforming growth factor β (TGFβ) is considered a canonical tumor suppressor that exerts profound control upon epithelial proliferation. Although cancer must evade TGFβ growth regulation, complete loss of TGFβ signaling competency is not universal because autocrine TGFβ promotes malignant phenotypes, such as invasion, and paracrine TGFβ has pro-tumorigenic effects on the tumor microenvironment. Some cancers, including colorectal cancer, pancreatic cancer, and head and neck squamous cell carcinoma (HNSC), exhibit genetic alterations of key pathway components, including somatic mutations of SMAD4 (mothers against decapentaplegic family member 4) and TGFBR2 (transforming growth factor beta receptor 2) (2). The conversion from tumor suppressor to tumor promoter is one of the paradoxes that have complicated the targeting of TGFβ in cancer therapy. A clearer understanding of its detrimental effects on cancer biology could provide an actionable rationale for TGFβ inhibition in cancer therapy.

One aspect of TGFβ biology that remains poorly understood is its role in genomic stability, which was initially recognized more than 25 years ago, for example, as disclosed in Glick et al., 1996. Transforming growth factor Beta1 suppresses genomic instability independent of a G1 arrest, p53, and Rb. Cancer Res. 56, 3645-3650. Over the last decade it has been established that TGFβ regulates the expression or function of key DNA repair proteins, including ATM (ataxia telangiectasia mutated), BRCA1 (breast cancer 1 gene), and LIG4 (DNA ligase 4), which are necessary for maintenance of genomic integrity, for example, as reviewed in Liu et al., 2019., Misrepair in context: TGFβ regulation of DNA repair. Front. Oncol. 9, 799.

Faulty DNA repair is a hallmark of cancer, and specific repair defects can provide the basis for response to precise therapies, for example, as disclosed in Ceccaldi et al., 2016. Repair pathway choices and consequences at the double-strand break. Trends Cell Biol. 26, 52-64 (2016). Moreover, key DNA repair effectors are attractive targets for drug development, which can be deployed in cancers with specific vulnerabilities, as evidenced by the success of poly(ADP-ribose) polymerase (PARP) inhibitors in BRCA1/2 mutant tumors, for example, as described in Lord and Ashworth, 2017. PARP inhibitors: Synthetic lethality in the clinic. Science 355, 1152-1158.

Insight is provided by human papilloma virus (HPV) positive HNSC, which exhibits striking sensitivity to standard of care genotoxic therapy with cisplatin and radiotherapy. The inventors of the present disclosure have previously demonstrated that loss of TGFβ competency in HPV-positive cancer in turn compromises the canonical DNA double strand break (DSB) repair pathways, homologous recombination repair (HR) and non-homologous end-joining (NHEJ), as described in Liu et al., 2018. Q. Liu, Subjugation of TGFβ signaling by human papilloma virus in head and neck squamous cell carcinoma shifts DNA repair from homologous recombination to alternative end joining. Clin. Cancer Res. 24, 6001-6014. Pharmaceutical TGFβ inhibition in HPV-negative cancer cells replicates the DNA repair defects exhibited by HPV-positive cancer cells and tumors. When classical DSB repair is defective, alternative end-joining (alt-EJ, also called microhomology-mediated end-joining) is thought to take over as a back-up repair, for example, as described in Sfeir and Symington, 2015. Microhomology-mediated end joining: A back-up survival mechanism or dedicated pathway? Trends Biochem. Sci. 40, 701-714, and in. Wood and Doublie, 2016. DNA polymerase theta (POLQ), double-strand break repair, and cancer. DNA Repair 44, 22-32. In support of this, Liu et al., 2018 demonstrated that alt-EJ is increased in HPV-positive cells, and in HPV-negative cells in which TGFβ signaling is blocked. DSB repair by alt-EJ is highly error-prone because it generates frequent genomic deletions and insertions with microhomologies at processed ends. Hence cells using alt-EJ are more sensitive to genotoxic chemotherapy or radiotherapy as described in Liu et al., 2018. It has also been observed that radiosensitivity is increased when TGFβ signaling is inhibited, as described in Bouquet et al., 2011. Transforming growth factor B1 inhibition increases the radiosensitivity of breast cancer cells in vitro and promotes tumor control by radiation in vivo. Clin. Cancer Res. 17, 6754-6765; Hardee et al., 2012, Resistance of glioblastoma-initiating cells to radiation mediated by the tumor microenvironment can be abolished by inhibiting transforming growth factor-β. Cancer Res. 72, 4119-4129; and Bouquet et al., 2014, Attenuation of the DNA damage response by TGFβ inhibitors enhances radiation sensitivity of NSCLC cells in vitro and in vivo. Int. J. Radiat. Oncol. Biol. Phys. 91, 91-99. Accordingly, defective TGFβ signaling may present a specific therapeutic opportunity.

The view that alt-EJ provides a survival mechanism in the face of classical DNA repair failure has spawned efforts to target its effector, polymerase theta θ (encoded by POLQ), an approach supported by the high POLQ expression in HR-deficient breast and ovarian tumors. More recently, experiments using alt-EJ and HR competition repair substrates demonstrated that alt-EJ can be used to repair 10-20% of DSB even in mammalian cells where both HR and NHEJ are available, as described in Truong et al., 2013. Microhomology-mediated end joining and homologous recombination share the initial end resection step to repair DNA double-strand breaks in mammalian cells. Proc. Natl. Acad. Sci. U.S.A. 110, 7720-7725, demonstrating that that Pol deletion compromises cell survival even when canonical DSB repair pathways are intact, as described in Feng et al., 2019. Genetic determinants of cellular addiction to DNA polymerase theta. Nat. Commun. 10, 4286-4286

Accordingly, the role of alt-EJ in cancer is apparently more complex than a simple back-up system. Accordingly, there is a need in the art for a deeper understanding of the significance of alt-EJ function in cancer. As well, there is a need in the art for method of assessing alt-EJ processes relevant to cancer, particularly in the context of readily implanted clinal platforms. There is additionally a need in the art for methods of directing appropriate therapies to cancer cells to exploit this potential target.

Additionally, because TGFβ responsiveness is modulated by complex genetic and epigenetic mechanisms and varies widely across human cancers, there is a need in the art for methods of measuring relevant TGFβ signaling processes that underlie cancer and status, including a need in the art for facile methods of performing such assessments that can be implemented on clinically relevant platforms. Further, there is a need in the art for methods of directing effective therapies to patients based on an understanding of TGFβ functions.

Lastly, there is a need in the art for methods of manipulating cancer sensitivity to selected treatments by interventions that exploit TGFβ and alt-EJ gene biology.

### SUMMARY OF THE INVENTION

The inventors of the present disclosure have developed a novel methodology for identifying cancer subjects that are highly amenable to certain treatments. Specifically, as disclosed herein, certain cancer cells comprise what will be referred to herein as a “DNA damage repair deficit” phenotype or “DDR deficit” phenotype. Cancer cells having the DDR deficit phenotype are unexpectedly more susceptible to certain treatments, including genotoxic treatments, PARP inhibition, and immunotherapies. Advantageously, this phenotype and its associated amenability to these enumerated treatments appears to be a pan-cancer phenomenon that is broadly found in many types of cancers.

In a first aspect, the scope of the invention encompasses methods and associated compositions of matter useful for assessing the DDR deficit phenotype in cancer cells. In one implementation, the scope of the invention encompasses a novel DDR deficit gene expression signature, which is a transcriptional signature indicative of the DDR deficit phenotype. The scope of the invention further encompasses novel diagnostic assay kits for assessing the DDR deficit phenotype in cancer cells, for example, by means of the DDR deficit transcriptional signature.

In a second aspect, the scope of the invention encompasses assessing the DDR deficit phenotype in cancer cells of a subject, such that appropriate therapies may be directed those subjects most likely to respond well. The scope of the invention encompasses a method of determining if the DDR deficit phenotype is present in cancer cells of a subject, and if present, administering a therapeutic treatment such as genotoxic treatment, PARP inhibition, or immunotherapy, to which the cancer cells are likely to be responsive.

In a third aspect, the inventors of the present disclosure have, as disclosed herein, discovered that cancer cells that do not have the DDR deficit phenotype can be induced to a DDR deficit state, sensitizing the cancer cells to the enumerated treatments. Accordingly, the scope of the invention further encompasses a method of treating cancer by a dual-treatment strategy wherein the first treatment induces the DDR deficit phenotype in cancer cells and the second treatment is a treatment which kills cancer cells made susceptible by the induction of the DDR deficit phenotype.

These and other aspects of the invention are next described in detail.

### BRIEF DESCRIPTION OF THE DRAWINGS

FIGS. 1A, 1B, and 1C. TGFβ signaling promotes therapeutic resistance by endorsing an effective DDR, whereas TGFβ inhibition erases this advantage. FIG. 1A: TGFβ signaling promotes HR and NHEJ, the two most accurate and effective DNA repair pathways. TGFβ signaling inhibits the error-prone alt-EJ repair. FIG. 1B: Cells in which TGFβ signaling is inhibited or intrinsically impaired are deficient in HR and NHEJ and resort to alt-EJ, which increases their sensitivity to therapy-induced DNA damage. FIG. 1C: Because alt-EJ depends on PARP1 activity, cells dependent on alt-EJ are highly sensitive to PARP inhibitor. Overall, TGFβ inhibition and PARP1 inhibition will synergize to compromise all three DNA repair pathways and maximize tumor cell kill.

FIGS. 2A, 2B, 2C, and 2D. Consensus clustering of GSEA gene sets across nearly 11,000 TCGA solid cancers. Sixteen cancers showed significant anti-correlation between the alt-EJ and TGFβ signatures. FIG. 2A: TGFβ upregulation vs. alt-EJ activation for Tenosynovial giant cell tumor (TGCT, −0.0.65), Thyroid cancer (THCA, −0.45), Skin Cutaneous Melanoma (−0.53), Lung squamous cell carcinoma (LUSC, −0.51). FIG. 2B: Uterine Corpus Endometrial Carcinoma (UCEC, −0.37), Head and Neck squamous cell carcinoma (HNSC, −0.40), Bladder Urothelial Carcinoma (BLCA, −0.34), and Ovarian serous cystadenocarcinoma (0V, −0.38). FIG. 2C: Pancreatic adenocarcinoma (PAAD, −0.08), Breast invasive carcinoma (BRCA, −0.46), Colon adenocarcinoma (COAD, −0.20), and Esophageal carcinoma (ESCA, −0.023). FIG. 2D: Glioblastoma multiforme, (GBM, −0.36), Prostate adenocarcinoma, (PRAD, −0.37), Lung adenocarcinoma, (LUAD, −0.09), and Liver hepatocellular carcinoma (LIHC, −0.028).

FIG. 3. FIG. 3 depicts a Kaplan-Meir survival curve showing that TGFβ and alt-EJ β-alt score associate with survival. High TGFβ and low alt-EJ was associated with increased survival.

FIG. 4. FIG. 4 depicts a Kaplan-Meir survival curve showing that TGFβ inhibition sensitizes SB28 murine glioma model tumors to radiotherapy at 10 Gy. Sham=treatment with control antibody; 1D11=treatment with 1D11 antibody inhibitor of TGFβ; RT=radiotherapy at 10 Gy, single dose; RT+1D11=combined radiotherapy with TGFβ inhibitor 1D11.

FIGS. 5A, 5B, and 5C. FIGS. 5A, 5B and 5C depict the association of β-alt signature with platinum and olaparib sensitivity. FIG. 5A: TGFβ signalling and alt-EJ activation ssGEA β-alt scores are negatively correlated. FIG. 5B: β-alt and cisplatin IC50 for ovarian cancer cell lines are negatively correlated. 5C: β-alt and olaparib IC50 for ovarian cancer cell lines are negatively correlated.

FIG. 6. FIG. 6 is a box plot depicting the ssGSEA TGFβ scores, the ssGSEA alt-EJ scores, and the β-alt scores for ovarian cancers found sensitive to cisplatin and resistant to cisplatin.

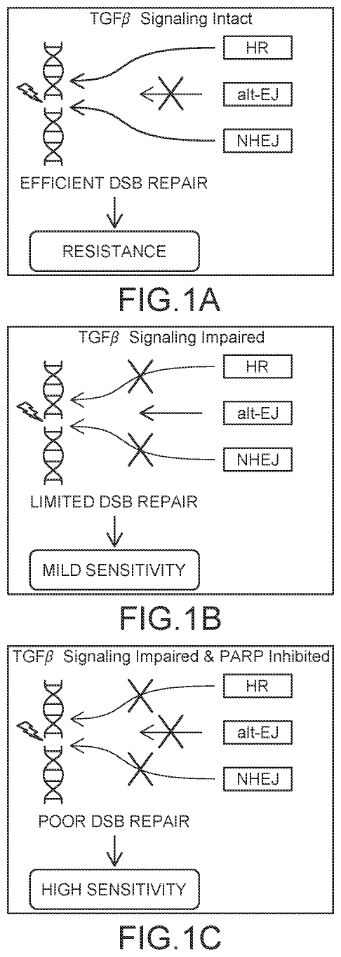

### DETAILED DESCRIPTION OF THE INVENTION

The inventions disclosed herein are based on the unexpected discovery that certain cancer cells have what is called herein the DDR deficit phenotype, and that such cancer cells are particularly susceptible to certain treatments. In cells, DNA damage is a regular occurrence that may result from various causes, including exposure to radiation or mutagenic agents, or errors in normal DNA replication. Eukaryotic cells cope with DNA damage by employing various repair mechanisms, commonly referred to as DNA damage repair or “DDR” mechanisms. Among the common DNA insults that cells may experience are double-strand breaks, commonly referred to as DSBs. Double strand breaks can be particularly harmful, causing genomic rearrangement, mutation, and cell death. Cells have three main DDR processes that can be engaged to repair DSBs: Homologous recombination repair (HR), non-homologous end-joining (NHEJ), and alternative end-joining (alt-EJ). HR and NHEJ are believed to be efficient processes that repair DSBs with little error. In contrast, Alt-EJ is known to be an error-prone repair mechanism, associated with insertion-deletion mutations (indels) and creating “genomic scars” at repair sites.

Transforming growth factor-beta, referred to as TGFβ, is a pleiotropic factor that is active in many cellular processes, including development, growth, differentiation, and cellular homeostasis. Among its many functions, it is believed that TGFβ signaling activity induces and maintains effective HR and NHEJ DDR processes in response to genomic stress. In cancer cells, rapid cell division causes severe genomic stress, and TGFβ signaling is of particular importance.

In some cancers, it has been observed that impairment of TGFβ signaling activity results in the loss of HR and NHEJ DDR processes and may increase the use of the error-prone, alt-EJ DNA DDR response, for example, as described in Liu 2018, Stefir 2015, and Wood 2016. As disclosed herein, the inventors of the present disclosure have discovered that cancer cells having the combination of impaired TGFβ signaling activity and increased alt-EJ activation are particularly sensitive to certain treatments. The DDR deficit phenotype encompasses cells having the combination of (1) reduced TGFβ signaling activity and (2) increased alt-EJ activity. This is considered a deficit in DDR capability because the efficient DNA damage repair by HR and NHEJ pathways is not available, while the inefficient alt-EJ process on which the cell must rely imparts a mutational burden and genetic instabilities, greatly reducing the ability of the cell to respond to and survive certain treatments.

In one aspect, cancer cells having the DDR deficit phenotype are more susceptible to genotoxic treatments, such as ionizing radiation or chemotherapeutic agents that induce double strand breaks. These treatments incur a mutational burden that cells having the DDR deficit phenotype are unable to overcome due to their deficit in DNA repair capability.

In another aspect, cancer cells having the DDR deficit phenotype are more susceptible to what is termed herein as PARP inhibition. Poly [ADP-ribose] polymerase 1 (PARP-1) is a critical regulator of DNA damage repair, facilitating repair by activating repair pathways and by its actions on chromatin and repair enzymes. PARP1 activity is necessary to alt-EJ activity. Inhibition of PARP1 pathways therefore will be harmful to cells that are reliant on alt-EJ for survival.

In another aspect, cells having the DDR deficit phenotype are susceptible to a variety of immunotherapy treatments. Without being bound to a particular theory of operation, it is believed that this susceptibility may arise from an increase in neoantigen production caused by the increased number of indels in cells reliant on alt-EJ, and/or may be the result accumulated mutations impairing the cells' ability to avoid immune surveillance.

Accordingly, there is a need to identify subjects having cancers with the DDR deficit phenotype, so that the effective treatments outlined above may be directed thereto. In one aspect, the scope of the invention encompasses a method of assessing DDR deficit phenotype in cancer cells. The general method of the invention encompasses the steps of:
obtaining a sample comprising cancer cells from a subject;
analyzing the cancer cells in the sample to determine if TGFβ signaling is impaired therein;
analyzing the cancer cells in the sample to determine if alt-EJ is activated;
wherein, if both impaired TGFβ signaling impairment and alt-EJ activation are observed in the cells, the cancer cells are determined to have the DDR deficit phenotype.

The analyses of both TGFβ signaling and alt-EJ active may be achieved by various methods. In a primary implementation, the analyses are achieved by measuring the expression of selected TGFβ signaling genes and alt-EJ genes that have been identified by the inventors of the present disclosure as indicative of the DDR deficit phenotype. The various elements and implementations of this general method are described next.

As used herein, a “subject” may be a human or a non-human animal such as a test animal or veterinary subject. As used herein, a “cancer subject” may be a subject having cancer, or at risk of having cancer, for example, a subject putatively having cancer, a subject diagnosed with cancer, or a cancer-free subject that has been previously treated for cancer.

As used herein, “cancer cells” may refer to cells of any neoplastic condition, including cancers such as carcinomas, sarcomas, or hematopoietic cancers. In one aspect, the cancer is a carcinoma. Carcinomas may comprise, for example, bladder cancer, brain cancer, breast cancer, cervical cancer, colorectal cancer, endometrial cancer, esophageal cancers, gastric cancer, glioblastoma, glioma, head and neck cancer, lung cancer, melanoma, mesothelioma, nasopharyngeal cancer, ovarian cancer, pancreatic cancer, prostate cancer, renal cancer, testicular cancer, thyroid cancer, skin cancer, and uterine cancer. In one embodiment, the cancer is a sarcoma selected from the group consisting of undifferentiated pleomorphic sarcoma, epithelioid sarcoma, liposarcoma, and leiomyosarcoma. In one embodiment, the cancer is a hematopoietic cancer selected from the group consisting of leukemia, lymphoma, and myeloma.

As used herein, a “sample” may encompass any cancerous tissue, including tumor cells, potentially cancerous tissue, or precancerous tissue. The cancer cells may comprise tumor cells, for example, primary tumor cells, cells from metastasis, circulating tumor cells, etc. Samples may be identified as cancerous by any means, including morphological markers, expression of molecular markers, staining patterns, or by their presence in a tumor, mass, lesion or other cancerous or cancer-like growth. Sample acquisition may be by means known in the art, including for example, tumor biopsy, such as a punch biopsy, fine needle aspiration biopsy, use of resected tumors, or other tumor tissue sampling methods. Samples may be analyzed in any format, including tissue sections, such as paraffin-embedded tissue sections (e.g. formalin-fixed paraffin-embedded (FFPE) tissue blocks), isolated cells, cultured cells derived from biopsy or explant material, or others.

As used herein, “a pharmaceutically effective amount” means an amount sufficient to induce a measurable biological and/or therapeutic effect.

In a primary embodiment, the assessment of TGFβ signaling competency and alt-EJ activation is achieved by measuring the expression of relevant genes. The inventors of the present disclosure have identified certain genes that are markers of TGFβ signaling processes that underlie maintenance and activation of the HR and NHEJ DDR mechanisms. Such genes may comprise genes that are active effectors of TGFβ signaling, or may be genes wherein the expression of the gene is otherwise correlated with the TGFβ signaling processes that underlie maintenance and activation of the HR and NHEJ DDR mechanisms. Such genes, comprising genes wherein the expression of the gene is positively correlated with TGFβ-mediated processes that maintain HR and NHEJ, will be referred to herein as “TGFβ-associated genes.”

Furthermore, the inventors of the present disclosure have identified certain genes that are markers of alt-EJ processes that underlie maintenance and activation of the alt-EJ DDR mechanism. Such genes may comprise genes that are active in such alt-EJ, or may be genes wherein the expression of the gene is otherwise correlated with maintenance and activation of the alt-EJ DDR mechanism. Such genes, comprising genes wherein the expression of the gene is positively correlated with alt-EJ activity, will be referred to herein as “altEJ-associated genes.”

In a primary embodiment, the DDR deficit phenotype is assessed in cancer cells of a sample by the following method:
obtaining a sample comprising cancer cells from a subject;
measuring the expression of one or more selected TGFβ-associated genes in the cancer cells of the sample;
measuring the expression of one or more selected altEJ-associated genes in the cancer cells of the sample;
wherein, low expression of the selected TGFβ-associated genes is indicative of impaired TGFβ signaling in the cancer cells;
wherein, high expression of the selected altEJ-associated genes is indicative of alt-EJ activation in the cancer cells; and
wherein, if both low expression of the selected and high expression of the selected altEJ-associated genes is observed, the cancer cells are deemed to have the DDR deficit phenotype.

A “panel” or grouping of one or more TGFβ-associated genes may be selected from the group consisting of:
Adhesion molecule with Ig like domain 2, gene name “AMIGO2”;
ATP binding cassette subfamily G member 1, gene name “ABCG1”;
Basic fibroblast growth factor, gene name “FGF2”;
C-C motif chemokine ligand 20, gene name “CCL20”;
Carbonic anhydrase 12, gene name “CA12”;
Coagulation factor III, gene name “F3”;
Collagen Type IV alpha 2 chain, gene name “COL4A2”;
Connective tissue growth factor, gene name “CTGF”;
Cytohesin-1, gene name “PSCD1”;
Deleted in Liver Cancer 1, gene name “DLC1”;
Desmocollin 2, gene name “DSC2”;
DNA binding protein inhibitor ID1, gene name “ID1”;
Dnaj subfamily B, member 9, gene name “DNAJB9”;
Ectoderm neural cortex protein 1, gene name “ENC1”;
Ectodermal neurocortex 1, gene name “ENC1”;
Fibroblast activation protein alpha, gene name “FAP”;
Fibroblast growth factor 2, gene name “FGF2”;
Fibronectin 1, gene name “FN1”
Glia-derived Nexin, gene name “SERPINE2”; Hes-related family BHLH Transcription factor with YRPW motif 1, gene name “HEY1”;
High mobility group AT-hook 2, gene name “HMGA2”;
Insulin Like Growth Factor 2 MRNA Binding Protein 3, gene name “IGF2BP3”;
Insulin-like growth factor binding protein 3, gene name “IGFBP3” Jagged 1, gene name “JAG1”;
Kruppel like factor 4, gene name “KLF4”;
La-regulated protein 6, gene name “LARP6
Laminin beta 3, gene name “LAMB3”;
Laminin subunit gamma-2, gene name “LAMC2”;
Lipase, endothelial type, gene name “LIPG”;
Maf BZIP Transcription Factor F, gene name “MAFF”;
Monocyte to macrophage Differentiation associated, gene name “MMD”;
Neural acetocholine receptor subunit alpha-9, gene name “CHRNA9”;
Periostin, gene name “POSTN”;
Plasminogen activator inhibitor-1, gene name “SERPINE1”;
Platelet derived growth factor C, gene name “PDGFC”;
Pleckstrin 2, gene name “PLEK2”;
Plexin A2, gene name “PLXNA2”;
Rho-GTPase-activating protein 32, gene name “RICS”;
Ring Finger Protein 24, gene name “RNF24”;
Runt-related Transcription Factor 1, gene name “RUNX1”;
SAM Domain SH3 Domain and Nuclear Localization Signals 1, gene name “SAMSN1”;
SH2 domain-containing protein 2A, gene name “SH2D2A”;
SH2 domain-containing protein 4A, gene name “SH2D4A”;
Solute Carrier Family 20, member 1, gene name “SLC20A1”;
Solute carrier Family 22, member 4, gene name “SLC22A4”;
Spindle apparatus coiled-coil domain containing Protein 1, gene name “CCDC99”; Tenascin C, gene name “TNC”;
TGFβ Induced Factor Homeobox 1; gene name “TGIF1”;
Thromobspondin 1, gene name “THBS1”;
Transmembrane Protein Androgen-induced Protein, gene name “TMEPAI”;
Tumor Necrosis Factor Receptor superfamily member 12A, gene name “TNFRSF12A”;
Versican, gene name “VCAN”

The panel may comprise any combination of one or more the aforementioned TGFβ-associated genes. In various embodiments, the panel comprises one, two, three, four, five, six, seven, eight, nine, ten, eleven, twelve, thirteen, fourteen, fifteen, sixteen, seventeen, eighteen, nineteen, twenty, 21, 22, 23, 24, 25, 26, 27, 28, 29, 30, 31, 32, 33, 34, 35, 36, 37, 38, 39, 40, 41, 42, 43, 44, 45, 46, 47, 48, 49, or all 50 of the enumerated TGFβ-associated genes. The panels may comprise additional TGFβ-associated genes not listed herein.

A panel or grouping of one or more altEJ-associated genes may be selected from the group consisting of:
Apurinic/Apyrimidinic Endodeoxyribonuclease 2, gene name “APE2” or “APEX2”;
Apurinic/Apyrimidinic Endodeoxyribonuclease 1; gene name “APE” or “APEX1”;
Anti-Silencing Function 1A Histone Chaperone; gene name “ASF1A”;
Cyclin Dependent Kinase Inhibitor 2D, gene name “CDKN2D”;
Calcium And Integrin Binding 1; gene name “CIB1”;
DNA Replication Helicase/Nuclease 2; gene name “DNA2”;
FA Core Complex Associated Protein 24; gene name “FAAP24”;
FA Complementation Group M; gene name “FANCM”;
Holliday Junction 5′ Flap Endonuclease GEN homolog 1: gene name “GEN1”;
HRas Proto-Oncogene, gene name “HARAS1”;
DNA Ligase 1; gene name “LIG1”;
DNA Ligase 3; gene name “LIG3”;
Menin 1; gene name “MEN1”;
Double-strand break repair protein MRE11; gene name “MRE11A”
DNA mismatch repair protein Msh3; gene name “MSH3”;
DNA mismatch repair protein Msh6; gene name “MSH6”;
Nudix Hydrolase 1; gene name “MTH1” or “NUDEX1”;
Mechanistic Target Of Rapamycin Kinase; gene name “MTOR”;
NSF Attachment Protein Beta; gene name “NAPB2”;
Nth Like DNA Glycosylase 1; gene name “NTHL1”;
Partner and localizer of the BRCA2 gene, gene name “PALB2”;
(Poly(ADP-Ribose) Polymerase 1, gene name “PARP1”;
(Poly(ADP-Ribose) Polymerase 3, gene name “PARP3”;
DNA Polymerase Alpha 1, Catalytic Subunit, gene name “POLA1”;
DNA Polymerase Mu, gene name “POLM”;
DNA Polymerase Theta, gene name “POLQ”;
Pre-MRNA Processing Factor 19, gene name “PRP19”;
Rad51 paralog D, gene name “RAD51D”;
RB Binding Protein 8, Endonuclease, gene name “RBBP8”;
Ribonucleotide Reductase Regulatory Subunit M2, gene name “RRM2”;
RuvB Like AAA ATPase 2, gene name “RUVBL2”;
Superoxide Dismutase type 1, gene name “SOD1”;
Histone Acetyltransferase Tip60, gene name “TIP60”;
Uracil DNA Glycosylase, gene name “UNG”;
WRN RecQ Like Helicase, gene name “WRN”; and
X-Ray Repair Cross Complementing 1, gene name “XRCC1”

The panel may comprise any combination of one or more the aforementioned altEJ-associated genes. In various embodiments, the panel comprises one, two, three, four, five, six, seven, eight, nine, ten, eleven, twelve, thirteen, fourteen, fifteen, sixteen, seventeen, eighteen, nineteen, twenty, 21, 22, 23, 24, 25, 26, 27, 28, 29, 30, 31, 32, 33, 34, 35, or all 36 of the enumerated altEJ-associated genes. The panels may comprise additional altEJ-associated genes not listed.

Certain of the TGFβ-associated and altEJ-associated genes are particularly highly indicative of the DDR deficit phenotype. The TGFβ-associated genes having high prognostic relevance are FAP, FN1, POSTN, SERPINE1, and THBS1. The altEJ-associated genes having high prognostic relevance are GEN1, RRM2, DNA2, POLQ, and LIG1. Accordingly, in some embodiments, the panels of TGFβ and alt-EJ associated genes will comprise one or more of these highly prognostic genes. In one embodiment, the selected TGFβ-associated genes of the panel will comprise one or more of FAP, FN1, POSTN, SERPINE1, and THBS1. In one embodiment, the selected TGFβ-associated genes of the panel will comprise FAP, FN1, POSTN, SERPINE1, and THBS1. In one embodiment, the selected altEJ-associated genes of the panel will comprise one or more of GEN1, RRM2, DNA2, POLQ, and LIG1. In one embodiment, the selected altEJ-associated genes of the panel will comprise GEN1, RRM2, DNA2, POLQ, and LIG1.

The aforementioned TGFβ-associated genes and altEJ-associated genes listed above are the human gene forms. It will be understood that the scope of the invention extends to non-human orthologs and homologs of the aforementioned genes, including for use in assaying the cancer cells of other species such as test animals. For example, the scope of the invention encompasses the murine, rat, zebrafish, drosophila, and other non-human versions of the enumerated genes.

In the methods of the invention, the determination of impaired TGFβ signaling in cells is made by the ascertainment of “low” expression of the selected genes. Low, in this context, may comprise a value that is below a selected threshold baseline value. The threshold value may comprise any suitable baseline, for example: the expression level of all genes measured in the assay; the expression level of all genes; the expression level of selected benchmark genes; the expression level of selected housekeeping genes; the expression level in like non-cancerous cells or other selected cell types, or any other measure known in the art for establishing a gene expression comparative baseline. In various implementations, low expression is expression that is at least 5%, at least 10%, at least 15%, at least 20%, at least 25%, at least 30%, at least 35%, at least 40%, at least 50%, at least 60%, at least 70%, at least 80%, or at least 90% below the selected threshold. In the case of multiple gene panels, the expression level of the various genes may be averaged or normalized by means known in the art to determine whether the measured values represents “low” gene expression by the panel as a whole.

Likewise, with regards to altEJ-associated genes, a determination of alt-EJ activation in the assayed cancer cells is made when the expression of the selected altEJ-associated genes is “high.” High expression, in this context, may comprise a value that is above a selected threshold baseline value. The threshold value may comprise any suitable baseline, for example: the expression level of all genes measured in the assay; the expression level of all genes; the expression level of selected benchmark genes; the expression level of selected housekeeping genes; the expression level observed in like non-cancerous or other selected cells; or any other measure known in the art for setting a gene expression comparative baseline. In various implementations, high expression is expression that is at least 5%, at least 10%, at least 15%, at least 20%, at least 25%, at least 30%, at least 35%, at least 40%, at least 50%, at least 60%, at least 70%, at least 80%, at least 90%; at least 100%, at least 200%, at least 300%, at least 400%, or at least 500%, at least ten times, at least 20 times, at least 50 times, or at least 100 times above the selected threshold. In the case of multiple gene panels, the expression level of the various genes may be averaged or normalized by means known in the art to determine whether the measured values represents “high” expression of the panel as a whole.

If both low TGFβ-associated gene expression and high alt-EJ activation associated gene expression is observed in cancer cells of the sample, the cancer cells are deemed to have the DDR deficit phenotype.

In various implementations, the determination of DDR deficit phenotype is assessed using an integrated score. The use of integrated scores is known in the art and any number of such scoring systems may be utilized by practitioners to provide a facile method of assessing DDR deficit phenotype. In a general implementation, the integrated score of the invention encompasses the development of equations that utilize measured TGFβ-associated genes and altEJ-associated genes expression data to generate a numeric value indicative of both the level of TGFβ signaling impairment and the level alt-EJ activation. This number may then be compared to a threshold value, standard curve, or other numeric guide to categorize sample cancer cells as having the DDR deficit phenotype, or other categories, such as “likely has DDR deficit phenotype” vs. “likely does not have DDR deficit phenotype”; “does not have DDR deficit phenotype” vs. “has mild DDR deficit phenotype” vs. “has severe DDR deficit phenotype,” etc. Integrated scores may use weighting coefficients for the various TGFβ-associated genes and altEJ-associated genes to improve resolution. Alternatively, each gene in the signature may be afforded equal weight in the calculation of the integrated score.

In one embodiment, the scope of the invention encompasses the use of a classifier model to determine DDR deficit status. For example, a classifier or predictive model generated using statistical methods such as: machine learning classifiers such as random forest, support vector machines, and newer deep learning and neural network approach and other statistical model generating methods known in the art. The output of the model may be a classification, score, or other output indicative of the assayed cancer cells risk or probability of having the DDR deficit phenotype or not.

In an exemplary embodiment, determination of DDR deficit status is assessed using an integrated score called herein the “β-alt score.” In a given cohort, the β-alt score is calculated as

βAlt scoreᵢ=\sqrt{(TGFβₘₐₓ−TGFβᵢ)²+(AltEjₘᵢₙ−AltEjᵢ)²}−\sqrt{(AltEjₘₐₓ−Altjᵢ)²+(TGFβₘᵢₙ−TGFβᵢ)²}
wherein:
   TGFβₘᵢₙ is Lowest value among all TGFβ scores;
TGFβₘₐₓ is Highest value among all TGFβ scores;
TGFβᵢ is The sample i TGFβ score;
AltEjₘᵢₙ is Lowest value among all AltEj scores;
AltEjₘₐₓ is Highest value among all AltEj scores; and
AltEjᵢ is The sample i AltEj score;
wherein an βAlt score above a selected threshold indicative of DDR deficit phenotype, is indicative of amenability to the selected treatment.
   The PAU score will range from −1.0 to 1.0, and, in one embodiment, a score above a defined threshold in the cohort or relative to a defined standard is indicative of the DDR deficit phenotype. Exemplary thresholds above which cancer cells of a sample are determined to have the DDR deficit phenotype include, scores above zero, for example, 0.1, 0.2, 0.3, 0.4, 0.5, 0.6, 0.7. 0.8, and 0.9.

The use of transcriptional signatures provides a facile and rapid means of assessing the DDR deficit phenotype that may be employed with clinically relevant platforms. However, it will be understood that the scope of the invention encompasses any method of assessing DDR deficit phenotype by measuring TGFβ signaling impairment and alt-EJ activation in cancer cells, including the use of methodologies other than gene expression biomarkers. Functional assays may be utilized instead of transcriptional data, or in combination with transcriptional data. For example, TGFβ signaling competency may be assessed by quantification of TGFβ in samples, for example by immunochemical detection of TGFβ or its effectors. Alternatively, functional assays for TGFβ activity may be used, for example, measuring the phosphorylation of Ataxia telangiectasia mutated (ATM), a TGFβ-regulated effector of DDR processes, for example, as described in Nyati et al., 2017. Quantitative and Dynamic Imaging of ATM Kinase Activity, Methods Mol Biol. 2017; 1596: 131-145 and Williams et al., 2013. Molecular imaging of the ATM kinase activity. Int J Radiat Oncol Biol Phys. 86:969-77. In another embodiment, TGFβ activity is assayed by measuring the phosphorylation state of SMAD2, for example by methods such as those described in Farrington et al., 2007. Development and validation of a phosphorylated SMAD ex vivo stimulation assay, Biomarkers 12:313-30 and Nyati et al., 2011. Molecular imaging of TGFβ-induced Smad2/3 phosphorylation reveals a role for receptor tyrosine kinases in modulating TGFβ signaling, Clin Cancer Res. 17: 7424-7439. Alternative assays for alt-EJ include measurement of unrepaired DNA damage as exemplified by the frequency of 53BP1 foci in cancer cells of the sample several hours following irradiation, for example, as described in Clin Cancer Research 24:6001-6014. Another method of measuring alt-EJ activity utilizes CRISPR-induced breaks followed by sequencing of the break sites to assess repair efficacy, for example, as described in Hussain et al., 2021 Measuring nonhomologous end-joining, homologous recombination and alternative end-joining simultaneously at an endogenous locus in any transfectable human cell, Nucleic Acids Research, gkab262.

Sequencing Methods and Assay Kits. The expression signatures of the invention may be assessed by any number of gene expression measurement techniques known in the art. The methods of the invention may be carried out by any sequencing platform.

In one embodiment, the quantification of TGFβ-associated genes and altEJ-associated genes is achieved by means of barcoded probes. Barcoded probe systems directly count the number of transcripts in a sample. These platforms utilize a set of nucleic acid probes specific for each transcript of the target genes, wherein the probes are conjugated to molecular barcodes. Barcodes may comprise heteropolymers of fluorescent moieties, wherein the order of the fluorescent moieties creates a unique identifier for each construct. These are hybridized to mRNA in the sample in solution phase, then hybridized probes are immobilized on a solid substrate and imaged by fluorescent microscopy to count the number of transcripts by reading of the barcodes. This methodology enables direct counting of transcripts without amplification or cDNA steps that can introduce bias. Exemplary barcode platforms include the NCOUNTER™ system by Nanostring Technologies (Seattle, WA, US).

Other transcript quantification methods include RNAseq Next Generation Sequencing technologies. In a general method, messenger RNA in the sample is fragmented and reverse transcribed into cDNA fragments. These are subsequently amplified and read by high throughput sequencing devices. Alternatively, mRNA in the sample may be read directly in some platforms. The use of random primers results in amplification of the entire transcriptome while targeted primers can be used to selectively amplify genes of interest. Exemplary RNAseq platforms include TEMP-O-SEQ™ (Bio-Spyder Inc., Carlsbad, CA, US) and ION APLISEQ™ (ThermoFisher, Waltham, MA, US).

In another implementation, the expression of the selected TGFβ-associated genes and altEJ-associated genes may be measured by use of a microarray, as known in the art. In this technique, the sample RNA is converted to cDNA, fluorescently labeled, and presented to an array of complementary nucleic acid probes specific for the transcripts of interest, immobilized on a solid support such as a chip or bead. Fluorescent signal is quantified to determine expression level. Exemplary microarrays for expression analysis include DYNABEAD™ (ThermoFisher, Waltham, MA, US) and Agilent arrays (Agilent, Santa Clara, CA, US).

In another implementation, the expression of the selected TGFβ-associated genes and altEJ-associated genes is measured using quantitative PCR (qPCR). mRNA in the sample is transcribed to cDNA, then amplified using primer pairs specific for the transcripts of interest, followed by quantification. Exemplary qPCR platforms include CFX OPUS™ (Bio-Rad, Hercules CA, US) and APPLIED BIOSYSTEMS™ qPCR platforms.

Assay Kits. The scope of the invention further encompasses assay kits that can be used to facilitate the facile and convenient assessment of DDR deficit phenotype in a sample. As used herein, an “assay kit” will refer to an aggregated collection of products that can be used to quantify two or more DDR deficit biomarkers of the invention in a sample. In one implementation, the assay kit will comprise a suite of two or more polynucleotide probes. Each polynucleotide probe will comprise a sequence that is complementary to and which will, under suitable conditions, hybridize to an mRNA or cDNA of a selected TGFβ-associated gene or altEJ-associated gene. Probes may comprise any complementary subsequence of the selected gene; probes may be engineered to increase specificity or stability; probes may be modified to contain sequences that increase detection sensitivity. In some embodiments, probe length is about 10-1,000 base pairs in length, for example, comprising about 50, 100, or 200 base pairs. For example, in the case of NANOSTRING™ type probes, probes of about 100 base pairs, preferentially complementary to the 3′ end of the target mRNAs are used. The probes will comprise a unique, distinguishable subsequence of the targeted cDNA or mRNA nucleic acid, selected for optimal hybridization depending on the sequencing platform. In some embodiments, the probes are immobilized on a substrate, such as a bead or planar biochip, as in a gene expression microarray.

The assay kits may further comprise polynucleotide probes for mRNAs or cDNAs of reference genes, such as housekeeping genes, as known in the art. The probes may comprise labels such as enzymatic, fluorescent, metal, radiolabel or chemiluminescent labels, for example, fluorescent protein polymers acting as barcodes, for the quantification of target species. The probes may further comprise conjugation moieties for the attachment of sequencing adapters, solid phase binding, or other functionalizations. The probes may comprise nucleic acid sequences for binding of primers to amplify the bound target. The probes may comprise DNA, PNA, or other nucleic acid compositions capable of hybridization to mRNAs or cDNAs. The kits may comprise elements such as reference standards, washing solutions, buffering solutions, reagents, printed instructions for use, and containers. The assay kits of the invention may comprise assay biochips or microfluidic devices for sample analysis. The assay kits may further encompass software, e.g. non-transitory computer readable storage medium comprising a set of instructions for operating a computer program which aids in carrying out the measurement and analysis of gene expression levels of the target genes.

In one implementation, the assay kit of the invention comprises: two or more polynucleotide probes, wherein each probe comprises a sequence that is complementary to and which will, under suitable conditions, hybridize to, an mRNA or cDNA of a target transcript, wherein, the assay kit comprises probes for one or more TGFβ-associated gene selected from the group consisting of: ABCG1, AMIGO2, CA12, CCDC99, CCL20, CHRNA9, COL4A2, CTGF, DLC1, DNAJB9, DSC2, ENC1, ENC1, F3, FAP, FGF2, FGF2, FN1, HEY1, HMGA2, ID1, IGF2BP3, IGFBP3, JAG1, KLF4, LAMB3, LAMC2, LARP6, LIPG, MAFF, MMD, PDGFC, PLEK2, PLXNA2, POSTN, PSCD1, RICS, RNF24, RUNX1, SAMSN1, SERPINE1, SERPINE2, SH2D2A, SH2D4A, SLC20A1, SLC22A4, TGIF1, THBS1, TMEPAI, TNC, TNFRSF12A, VCAN; and probes for one or more altEJ-associated genes selected from the group consisting of: APE2, APEX1, ASF1A, CDKN2D, CIB1, DNA2, FAAP24, FANCM, GEN1, HARAS1, LIG1, LIG3, MEN1, MRE11A, MSH3, MSH6, MTH1, MTOR, NAPB2, NTHL1, PALB2, PARP1, PARP3, POLA1, POLM, POLQ, PRP19, RAD51D, RBBP8, RRM2, RUVBL2, SOD1, TIP60, UNG, WRN, and XRCC1.

In another implementation, the scope of the invention encompasses two or more PCR primer pairs for the selective amplification of mRNA and/or cDNA sequences of the target transcripts. In one embodiment, the assay kit of the invention comprises:
two or more PCR primer pairs, wherein each primer pair comprises two single-stranded oligonucleotide PCR primers, the two primers being of sequence selected to hybridize with an mRNA or cDNA of a selected transcript, and to enable, under suitable conditions, PCR amplification of sequences found on the target transcript, the target transcript being a transcript of a target gene;
wherein the assay kit comprises primer pairs for the amplification of one or more TGFβ-associated genes selected from the group consisting of: ABCG1, AMIGO2, CA12, CCDC99, CCL20, CHRNA9, COL4A2, CTGF, DLC1, DNAJB9, DSC2, ENC1, ENC1, F3, FAP, FGF2, FGF2, FN1, HEY1, HMGA2, ID1, IGF2BP3, IGFBP3, JAG1, KLF4, LAMB3, LAMC2, LARP6, LIPG, MAFF, MMD, PDGFC, PLEK2, PLXNA2, POSTN, PSCD1, RICS, RNF24, RUNX1, SAMSN1, SERPINE1, SERPINE2, SH2D2A, SH2D4A, SLC20A1, SLC22A4, TGIF1, THBS1, TMEPAI, TNC, TNFRSF12A, and VCAN; and
wherein the assay kit comprises primer pairs for the amplification of one or more altEJ-associated genes selected from the group consisting of: APE2, APEX1, ASF1A, CDKN2D, CIB1, DNA2, FAAP24, FANCM, GEN1, HARAS1, LIG1, LIG3, MEN1, MRE11A, MSH3, MSH6, MTH1, MTOR, NAPB2, NTHL1, PALB2, PARP1, PARP3, POLA1, POLM, POLQ, PRP19, RAD51D, RBBP8, RRM2, RUVBL2, SOD1, TIP60, UNG, WRN, and XRCC1.

The primers may comprise primers of any length suitable for PCR amplification of target sequences, for example, being 15-40 base pairs in length. Primers may comprise DNA, PNA, or other nucleic acid compositions capable of hybridization to mRNAs or cDNAs. The primer sequences may be selected using any number of methods known in the art for primer design, such as Primer-BLAST (available at https://www.ncbi.nlm.nih.gov/tools/primer-blast/) or OLIGO (for example, available at https://www.oligo.net/downloads.html).

Combined Diagnostic and Treatment Method. The methods and assay kits disclosed above provide the art with tools for assessing the DDR deficit phenotype. As set forth above, the inventors of the present disclosure have advantageously determined that cancer cells having the DDR deficit phenotype are particularly amenable to certain treatments, including genotoxic treatments, PARP inhibition, and immunotherapy. This discovery enables patient stratification and personalized medical treatment, wherein subjects may be directed to efficacious treatment options while avoiding the expense, side effects, and other risks of ineffective or incompatible treatments.

Accordingly, in a first implementation, the scope of the invention encompasses a method of identifying subjects having cancer cells that have the DDR deficit phenotype, and if a subject is found to have cancer cells with the DDR deficit phenotype, a treatment suitable for such cancer cells is administered. In one embodiment, the scope of the invention encompasses a method of selecting patients who will respond well to genotoxic treatment, by assessing the DDR deficit phenotype in cancer cells of the subject, wherein if the cancer cells are determined to exhibit the DDR deficit phenotype, the subject is deemed amenable to a genotoxic treatment. In one embodiment, the scope of the invention encompasses a method of selecting patients that will respond well to PARP inhibition, by assessing the DDR deficit phenotype in cancer cells of the subject, wherein if the cancer cells are determined to exhibit the DDR deficit phenotype, the subject is deemed amenable to a treatment with PARP1 inhibitors. In one embodiment, the scope of the invention encompasses a method of selecting patients that will respond well to immunotherapy treatment, by assessing the DDR deficit phenotype in cancer cells of the subject, wherein if the cancer cells are determined to exhibit the DDR deficit phenotype, the subject is deemed amenable to an immunotherapy treatment.

In one implementation, the scope of the invention encompasses a method of treating cancer in a subject in need of treatment therefor, comprising the steps of
assessing DDR deficit phenotype in cancer cells of the subject, wherein the DDR deficit phenotype comprises impaired TGFβ signaling and alt-EJ activation; and
wherein, if the cancer cells of the subject are determined to have the DDR deficit phenotype, the subject is administered one or more treatments selected from the group consisting of a genotoxic therapy, PARP inhibition, or an immunotherapy.

In various embodiments, the cancer cells may comprise cells of a carcinoma, sarcoma, or hematopoietic cancer. In some embodiments, the cancer is a carcinoma selected from the group consisting of bladder cancer, brain cancer, breast cancer, cervical cancer, colorectal cancer, endometrial cancer, esophageal cancers, gastric cancer, glioblastoma, glioma, head and neck cancer, lung cancer, melanoma, mesothelioma, nasopharyngeal cancer, ovarian cancer, pancreatic cancer, prostate cancer, renal cancer, testicular cancer, thyroid cancer, skin cancer, and uterine cancer. In some embodiments, the cancer is a sarcoma selected from the group consisting of undifferentiated pleomorphic sarcoma, epithelioid sarcoma, liposarcoma, and leiomyosarcoma. In some embodiments, the cancer is a hematopoietic cancer selected from the group consisting of leukemia, lymphoma, and myeloma.

In some embodiments, the subject is administered a genotoxic treatment. Genotoxic treatments that induce DSBs are particularly effective, however treatments that induce single-strand breaks or other types of DNA damage may be used as well. In a first implementation, the genotoxic treatment comprise the administration of a therapeutically effective amount of a genotoxic agent. As used herein, a therapeutically effective amount is an amount sufficient to produce a measurable biological or therapeutic effect.

The genotoxic agent may comprise any genotoxic agent known in the art, for example, any of alkylating agents, intercalating agents, topoisomerase poisons, and others known in the art. Exemplary genotoxic agents include, for example, platinum drugs such as cisplatin, carboplatin, and oxaliplatin; antimetabolites such as 5-Fluorouracil, fludarabine and methotrexate; alkylating agents such as temozolomide, MNNG, and dacarbazine; nitrogen mustards, such as chlorambucil and cyclophosphamide; and topoisomerase poisons, such as camptothecin based drugs and etoposide. Additional examples of genotoxic agents include 1,3-bis(2-chloroethyl)-1-nitrosourea (BCNU), busulfan, carmustine, chlorambucil, cyclophosphamide, dacarbazine, daunorubicin, doxorubicin, epirubicin, idarubicin, ifosfamide, irinotecan, lomustine, mechlorethamine, melphalan, mitomycin C, mitoxantrone, temozolomide, and topotecan.

In other embodiments, the genotoxic treatment comprises the administration of a therapeutically effective amount of ionizing radiation to the cancer cells of the subject. In one embodiment, the ionizing radiation is administered as an external beam therapy, as known in the art. Exemplary external beam therapies include X-rays, gamma rays (for example, as delivered by Cobalt-60 devices), high energy electrons (for example, as delivered by linear accelerators), proton beams, high linear energy transfer particles, and neutron beams.

In one embodiment, the administration of ionizing radiation is achieved by administration of a therapeutically effective amount of a radiopharmaceutical agent. A radiopharmaceutical agent is a composition of matter comprising a radioisotope that may be introduced to the body to deliver ionizing radiation to target tissues or organs. The radioisotope may be any known in the art, for example, a β-emitter such as Sumarium-153, Lutetium-177, Yttrium-90, Iodine-131; or an alpha-emitter such as Astatine-211, Actinium-225, Bismuth-213, Bismuth-212, Radium-223, Thorium-227, and Lead-212. In some implementations, the radiopharmaceutical comprises the radionucleotide delivered by itself. In various implementations, the radionucleotide is integrated within or conjugated to a delivery moiety for targeted or improved delivery. Exemplary delivery moieties include antibodies (for example, anti-CD33 antibodies such as lintuzumab, anti-CD38 antibodies, anti-CD20 antibodies such as rituximab, and anti-HER2/neu antibodies), peptides (for example, somatostatin analog peptides and octreotide), small molecules (for example, iobenguane 1-131, γ-glutamyl folic acid derivatives and the neuropeptide N-acetylaspartylglutamate), liposomes, nanoconstructs, and glass or resin microspheres.

In some embodiments, the ionizing radiation is delivered by use of a brachytherapy implant. Brachytherapy implants include radioactive “seed” bodies, pellets, or wires. Brachytherapy implants may comprise any suitable radiation source, for example, Cesium-131, Cesium-137, Cobalt-60, Iridium-192, Iodine-125, Palladium-103, Ruthenium-106, Radium-226. Typical brachytherapy targets include the prostate, breast tissue, esophagus, head and neck, cervix, and uterine tissues.

In other embodiments, the genotoxic treatment comprises the administration of a therapeutically effective amount of ultraviolet radiation to the cancer cells of the subject. In one embodiment, the ultraviolet radiation is administered as an external exposure, as known in the art. In other embodiment, ultraviolet radiation is delivered by a source to deep-seated tumors sometimes in conjunction with surgery

In some embodiments, the genotoxic treatment comprises a combination of genotoxic agents. For example, chemotherapy cocktails are known in the art, including additive combinations, potentiating combinations, and synergistic combinations. Additionally, the genotoxic treatment of the invention may comprise the administration of a combination of one or more chemotherapy agents with radiotherapy, chemoradiation combinations that are known in the art.

PARP Inhibition. Poly [ADP-ribose] polymerase 1 (PARP-1) is a critical regulator of DNA damage repair, facilitating the repair by activating repair pathways and by its actions on chromatin and repair enzymes. PARP1 activity is crucial to alt-EJ activity. Because cells having the DDR deficit are reliant on alt-EJ, inhibition of PARP1 is especially effective against such cells. If PARP1 is inhibited and if other repair pathways are unavailable, the cell is less likely to survive.

Accordingly, in one embodiment, the scope of the invention encompasses the treatment of a subject for cancer, the treatment comprising the steps of: assessing the DDR deficit phenotype in cancer cells of the subject, wherein, if the cancer cells of the subject are determined to have the DDR deficit phenotype, the subject is administered a pharmaceutically effective amount of a PARP inhibitor. In various embodiments, the PARP inhibitor is selected from the group consisting of: olaparib, rucparib, niraparib, talazoparaib, veliparib, pamiparib, AG1436,10EP 9722, E7016, 3-aminobenzamide, and BGB-290.

Immunotherapy. Immunotherapy seeks to activate the patient's immune system to eliminate cancer cells. To do so, the immune system must recognize aberrant cells. Cells having the DDR deficit phenotype are unable to efficiently repair DNA. The alt-EJ process upon which they are reliant results in numerous deletions and insertions. These mutagenic factors increase the likelihood of proteins having amino acid substitutions, protein truncations, and other irregularities. Such irregularities in a cancer cell's genome result in an increased probability for the formation of neoantigens, novel protein motifs displayed on the cancer cell surface that are recognized by immune surveillance and activate immune responses. Accordingly, cancer cells with the DDR deficit phenotype are more likely to have detectable neoantigens. In contrast, in cancers in which TGFβ is functioning normally, fewer neoantigens will be present. In one embodiment, immunotherapies seek to promote T cell cytotoxicity and rely on the presence of neoantigens that are more likely when cancer cells have a DDR deficit phenotype.

A variety of cancer immunotherapy treatments are currently being deployed or in development, acting by diverse effectors and pathways. By these immunotherapy treatments, immune system ability to target and destroy cancer cells is enhanced. Accordingly, in one aspect, the scope of the invention encompasses the administration of a therapeutically effective amount of an immunotherapy agent to treat cancer in a subject, wherein the cancer cells of the subject have been determined to have the DDR deficit phenotype.

Examples of immunotherapies include immune checkpoint inhibitors. Exemplary immune checkpoint inhibitors include, for example, inhibitors of CTLA-4, for example, Ipilimumab; inhibitors of PD-1, for example, Nivolumab and Pembrolizumab; and inhibitors of PD-L1, for example Atezolizumab, Avelumab, and Durvalumab.

In one embodiment, the immunotherapy is a cellular immunotherapy agent, such as dendritic cells that have been primed ex-vivo (e.g. Sipuleucel-T), chimeric antigen receptor T-cells (e.g., Tsagenlecleucel and axicabtagene ciloleucel), and tumor-infiltrating lymphocytes primed ex-vivo.

In one embodiment, the immunotherapy agent is an immunotherapy comprising an agent which primes immune cells in vivo, including: viral constructs that target tumor cells to express antigens or cytokines that stimulate the immune system; a tumor cell lysate; or an antigen-bearing antibody targeted to immune cells such as dendritic cells.

In one embodiment, the immunotherapy agent is a cytokine, such as interferon-alpha, interleukin-2, or GM-CSF.

In one embodiment, the immunotherapy agent is an antibody or antibody-drug conjugate directed to a cancer-associated antigen.

In one embodiment, the immunotherapy agent is a means to neutralize cytokines or growth factors that suppress immunity, such as TGFβ, interleukin 10 or interferon gamma.

Sensitizing Cancer Cells By Induction of the DDR Deficit Phenotype. As disclosed herein, cancer cells having the DDR deficit are more amenable to certain treatments such as genotoxic treatments, PARP inhibition, and immunotherapy. Advantageously, the inventors of the present disclosure have determined that the DDR deficit phenotype may be induced in the cancer cells of a subject, making them more amenable to these treatments. In one embodiment, the DDR deficit phenotype may be induced by inhibition of TGFβ signaling, resulting in the loss of HR and NHEJ repair pathways. This discovery provides the art with a means of inducing synthetic lethality to genotoxic treatments, PARP inhibition, and immunotherapy.

Accordingly, in one aspect, the scope of the invention encompasses a method of treating cancer in a subject in need of treatment therefor by the steps of:
administering to the subject a first treatment to induce a DDR deficit phenotype; and
administering to the subject one or more additional treatments that is effective in killing cancer cells having the DDR deficit phenotype.
   In a related embodiment, the scope of the invention encompasses:
A TGFβ inhibitor, for use in a method of treating cancer in a subject,
wherein the method of treating cancer comprises administering to the subject a TGFβ inhibitor; and
administering to the subject one or more additional treatments, wherein the one or more additional treatments comprises a treatment a that is effective in killing cancer cells having the DDR deficit phenotype;
   in some embodiments, the one or more additional treatments comprises a treatment that is effective in killing cancer cells having the DDR deficit phenotype comprises a treatments selected from the group consisting of a genotoxic treatment, PARP inhibition, and an immunotherapy.

In one implementation, DDR deficit is induced in the cancer cells of a subject by inhibiting TGFβ signaling activity therein. Inhibition of TGFβ signaling may be achieved by administration of a pharmaceutically effective amount of one or more TGFβ inhibitors. TGFβ inhibitors include any composition known in the art which interferes with the expression, translation, activity, and/or regulatory functions of TGFβ. TGFβ signaling inhibitors encompass any number of agents, such as neutralizing antibodies, ligand traps, kinase inhibitors, antisense compositions, and others that interfere with TGFβ signaling by various means, such as reducing TGFβ bioavailability, interrupting TGFβ-receptor interaction, and inhibiting TGFβ kinase functions. Ligand traps include activin based ligand traps, antibodies against TGFβ ligands and receptors, and agents such as Sotatercept and Luspatercept. Exemplary TGFβ inhibitor agents include: human monoclonal antibody, 264RAD; fresolimumab (GC1008); a human monoclonal antibody neutralizing TGFβ1; LY3022859; an anti-TβRII monoclonal antibody that inhibits receptor-mediated TGFβ signaling activation; galunisertib, a TβRI kinase inhibitor; Belagenpumatucel-L; gemogenovatucel-T, trabedersen, XOMA089; SB-431542; SB-545344; SB-505124; LY2109761; LY364947; LY2157299, IN-1130; SD-208; R-268712; A-7701; A-83-01; GW788388; pirfenidone; fluorofenidonel; CJJ300; Sotatercept; and Luspatercept. In some embodiments, two or more TGFβ signaling inhibitors are applied in combination.

In one implementation, DDR deficit is induced in the cancer cells of a subject by inhibiting the molecule by which TGFβ signaling suppresses altEJ. Inhibition of the molecule may be achieved by administration of a pharmaceutically effective amount of one or more inhibitors that include any composition known in the art which interferes with the expression, translation, activity, and/or regulatory functions. Molecular inhibitors encompass any number of agents, such as neutralizing antibodies, ligand traps, kinase inhibitors, antisense compositions, and others that interfere with the target by various means, such as reducing bioavailability, interrupting molecular interactions, and inhibiting kinase functions. These molecular targets are the mRNA or encoded proteins of genes that include Hypoxia Induced Factor 1 (gene name HIF1alpha) and Notch Receptor 1 (gene name NOTCH), or genes CDK7, ZNF143, MYC, ETS1, GABPA, RBPJ, MYCN, YY1, among others.

The second treatment may be any treatment that is likely to be effective against cancer cells having DDR deficit phenotype. In one embodiment, the suitable treatment is a genotoxic treatment. In one embodiment, the treatment is PARP1 inhibition. In one embodiment, the suitable treatment is an immunotherapy.

The timing of administration of the first, TGFβ inhibition treatment and the second selected treatment may be contemporaneous, sequential, or alternating. In a primary embodiment, the first and second treatments are applied contemporaneously, i.e. simultaneously or overlapping in time. In one embodiment, the first and second treatments are administered in combination product as a single dosage form.

### EXAMPLES

### Example 1. Pan Cancer Analysis of TGFβ Signaling and Alt-EJ Activation

effects in cancers. A vast literature on TGFβ biology in cancer indicates that it is key to many aspects of tumor biology, from growth control to vascularity, extracellular matrix composition and immune infiltrate yet the context in which TGFβ activity is clinically actionable has yet to be established.

HR and NHEJ are thought to be backed-up by alt-EJ in that failure of either process can increase deployment of alt-EJ. Thus, increased use of alt-EJ could be considered a consequence of defective HR. If so, restoration of BRCA1 should rescue HR and suppress alt-EJ. To test this, miR-182 was antagonized in TGFβ competent SAS cells, and the effect of TGFβ inhibition on HR and alt-EJ function was measured using pathway specific reporters for HR (pDRGFP), distal end-joining by either-NHEJ or alt-EJ (pimEJ5GFP) or alt-EJ (EJ2GFP). Cells expressing the miR-182 antagomir were HR competent when TGFβ was inhibited, consistent with the necessity of BRCA1 for HR competency; however, alt-EJ repair remained significantly increased upon TGFβ inhibition. This observation demonstrates for the first time that HR deficiency is not required for alt-EJ to increase when TGFβ is inhibited.

Next it was sought to test whether TGFβ inhibition would increase alt-EJ in the context of NHEJ inhibition. Here pulsed-field gel electrophoresis was used to measure residual DSB levels in SAS cells at various timepoints after irradiation (20 Gy). NHEJ was blocked by treating cells with the DNA-protein kinase inhibitor KU57788 1 hour before irradiation and TGFβ signaling was blocked 24 hours beforehand with LY36494 pretreatment. As expected, KU57788 significantly inhibited repair of radiation induced DSB, and was partially rescued by pretreatment with LY36494. This novel observation demonstrates that TGFβ inhibition promotes an alternative process of repair. Together, these data show that TGFβ signaling is essential for both the fundamental molecular mechanisms of DNA repair, i.e. ATM kinase activity, and the functional consequences such as DNA repair pathway choice and resolution of DSB.

TGFβ regulates expression of DDR genes. To further investigate TGFβ impact on DDR, the expression of DNA repair-associated genes was evaluated using the NANOSTRING™ DDR gene panel. Treatment of SAS cells for 24 h with TGFβ plus or minus its inhibitor LY2157299, revealed striking reciprocal regulation of 180 DDR genes. According to KEGG pathway analyses, expression of genes implicated in HR and NHEJ was increased by TGFβ and reduced by its inhibition. Consistent with prior literature, CDKNJA was strongly induced by TGFβ and blocked by LY2157299, even though SAS cells, like most cancer cells, are insensitive to TGFβ-mediated cell cycle control. BRCA1 expression was increased by TGFβ and suppressed by LY2157299, as was ABL1 and POLD4. In contrast, TGFβ decreased and LY2157299 inhibition increased expression of LIG1, PARP1, and POLQ, which are key genes involved in alt-EJ.

Given that miR-182 was essential for TGFβ-mediated control of HR and microRNAs can target hundreds of genes, we next determined whether miR-182 was involved in TGFβ regulated DDR gene expression. SAS cells in which miR-182 was overexpressed or antagonized were treated as above prior to analysis using the NANOSTRING™ panel. TGFβ-mediated changes in BRCA1 expression were miR-182 dependent, as previously reported, as were its effects on MRE11A, MYD88, and PARP3 expression. In contrast, changes in CCND2, CDKNJA, and POLD4 expression were miR-182-independent, consistent with the presence of SMAD binding elements in these genes. Notably, expression of the alt-EJ genes LIG1, PARP1, and POLQ was found to be miR-182-independent. Together these data confirm that TGFβ has a broad impact on DDR via expression and molecular regulation of many genes and via ATM kinase activity. They also extend the range of TGFβ influence on expression of DDR-associated genes, and show that this occurs through both miR-182-dependent and -independent mechanisms. As neither alt-EJ execution nor expression of critical genes in this process are miR-182-dependent, these data mechanistically separate the effects of TGFβ on HR from those on alt-EJ.

LIG1, PARP1, and POLQ do not contain recognizable SMAD-regulation elements yet their expression was decreased upon exposure to TGFβ. To confirm this effect, quantitative gene expression measurements were conducted as a function of duration of TGFβ stimulation or small molecule receptor kinase inhibition in SAS cells. Notably, expression of each of the three genes was reciprocally suppressed by TGFβ signaling and increased by its inhibition. Although the early (5 hour) regulation of POLQ is consistent with direct transcriptional regulation, the later effects on LIG1 and PARP1 are suggestive of indirect effects.

The above observations led to the hypothesis that TGFβ suppression of alt-EJ gene expression is a distinct mechanism that influences differential DNA repair pathway use. To test whether this biology is broadly employed beyond HNSC, glioblastoma (GBM) was considered because high TGFβ ligand and receptor expression correlate with poorer survival, and because TGFβ inhibition enhances radiosensitivity in GBM cell lines and primary tumor explants. First, the U251 human GBM cell line was investigated using the NANOSTRING™ panel. As observed in HNSC SAS cells, TGFβ inhibition increased expression of the key alt-EJ genes, LIG1, PARP1, and POLQ in GBM cells. These results were validated by quantitative expression assays in response to a time course of TGFβ treatment and TGFβ signaling blockade with LY2157299. As in HNSC cells, expression of LIG1, PARP1, and POLQ were significantly decreased upon TGFβ treatment, and increased when TGFβ signaling was blocked. U251 reporters (EJ2GFP) were established to evaluate alt-EJ repair. Consistent with SAS data, both of two specific TGFBR2 inhibitors markedly increased alt-EJ events, effects that were again independent of miR-182 status.

To interrogate more extensively the interplay between TGFβ and alt-EJ, a 36-gene alt-EJ competency signature was devised. This signature was evaluated in concert with a previously described 50-gene set that is induced by chronic TGFβ stimulation. There were no known targets of TGFβ in the alt-EJ signature gene list, nor vice versa. Unsupervised clustering of HNSC TCGA data using the chronic TGFβ signature had previously shown HPV-positive cancers to be TGFβ unresponsive. Here it was found that they are also characterized by high alt-EJ gene expression. Given that HPV-positive cancers were clustered with low expression of TGFβ target genes and high expression of alt-EJ genes, a single specimen gene set enrichment analysis (ssGSEA) was performed to determine the signature correspondence across TCGA HNSC specimens. Consistent with the biology described above, TGFβ and alt-EJ signatures showed a striking negative correlation (Pearson's correlation coefficient (PCC)=−0.4; P<0.00001. A significant negative correlation remained after removing HPV-positive cancers indicating that the relationship exhibited is also present in HPV-negative cancers. Next, ssGSEA scores of both signatures were used to examine unsupervised clustering of GBM TCGA microarray data. GBM patients clustered into two major arms, one characterized by low alt-EJ signature and one by relatively high alt-EJ score; of these a subset was associated with reciprocal expression of the TGFβ signature (PCC=−0.35; P=0.00001). Consistent with the biology observed in cell lines, these analyses reveal a strong reciprocal relationship between TGFβ competency and alt-EJ gene expression in human cancer.

Low TGFβ/high alt-EJ signature predicts better outcomes after genotoxic therapy. The alt-EJ repair process is both error-prone resulting in more genome alterations and less efficient such that cells using this pathway have greater sensitivity to DSB inducing agents. Consistent with this, Tgfb 1-null murine cells are genomically unstable, as are human cells in which TGFβ signaling is inhibited. Loss of TGFβ signaling, whether through HPV infection, ligand neutralizing antibodies or TGFβ receptor kinase inhibitors, increases sensitivity to DSB induced by ionizing radiation and platinum drugs. Because genotoxic therapy is standard-of-care (SOC) for many cancers, we postulated that patients with tumors characterized by low TGFβ and high alt-EJ signatures (TGFβ^{lo}/alt-EJ^{hi}) would have more genome alterations and be more responsive to genotoxic therapy than those with high TGFβ and low alt-EJ signatures (TGFβ^{hi}/alt-EJ^{lo}).

To classify patients according to their TGFβ and alt-EJ transcriptional profiles, a score was calculated (βalt; as described above) based on the difference between the TGFβ and alt-EJ normalized signature value in each cancer setting. The association between βalt and the fraction of tumor genome altered was estedfor all patients, and between βalt and overall survival (OS) and progression-free (PFS) or disease-free survival (DFS) for patients who were treated with genotoxic agents. Patient outcome was assessed by comparing the upper (i.e. TGFβ^{lo}/alt-EJ^{hi}) and lower (i.e. TGFβ^{hi})/alt-EJ^{lo}) βalt tertiles using the integrated pan-cancer clinical data resource.

To analyze the signatures in GBM, first tumors initially categorized as ‘neural’ were excluded since this subtype may represent samples contaminated by normal brain tissue, resulting in 442 cases. Again, both signatures were significantly anti-correlated in GBM (PCC=−0.35; P<0.00001). Although GBM generally exhibit low somatic mutation burden, the fraction of genome altered was significantly associated with βalt (Mann-Whitney test; P<0.001). SOC treatment for newly-diagnosed GBM compromises surgery, radiotherapy (RT) and chemotherapy (ChT) with temozolomide. To evaluate patient survival, datasets were curated to eliminate specimens from patients who were not treated with both RT and ChT, leaving a total of 274 cases. OS (log-rank test p=0.031) and PFS (log-rank test P=0.096) of patients with a high βalt score were greater than those with a low score. Hypermethylation of the MGMT promoter and mutation of IDH1/2 (37) are known prognostic biomarkers in GBM. Few TCGA specimens had IDH1/2 mutations (n=8). A multivariate Cox regression analysis was performed including MGMT status. This maintained the βAlt score association with OS (hazard ratio=0.70, 95% CI 0.96-0.51, p=0.026) and PFS (hazard ratio=0.66, 95% CI 0.89-0.49, p P=0.006), indicating that TGFβ and alt-EJ signatures are independent of MGMT status.

Patients with lung squamous cell carcinoma (LUSC) are generally treated with surgery, RT and/or ChT depending on tumor stage and lung function. LUSC also exhibited significant βalt anti-correlation (PCC=−0.43; P<0.00001) and a greater fraction of the genome altered was significantly correlated with high βalt scores (Mann-Whitney test, P<0.00001). Patients in whom it was specified that neither ChT nor RT had been given were excluded, as were stage I patients because they are usually treated with surgery alone. Based on SOC, the remaining patients (n=231) were likely to have been treated with ChT, RT or both. Thus, OS was better in patients with high βalt scores (log-rank test p=0.05), and PFS of these patients was also significantly increased (log-rank test p=0.02).

The anti-correlation of TGFβ and alt-EJ signatures in ovarian cancer (OVCA; n=541) was also significant (PCC=−0.32; P<0.00001) and the fraction of altered genome was greater in those with higher βalt scores (Mann-Whitney test, P<0.00001). Patients with stage II-IV serous ovarian cancer in the TCGA data set were treated with surgical resection followed by systemic treatment with platinum and taxane genotoxic agents. Compared to those with low βalt scores, both OS (log-rank test p=0.004) and PFS (log-rank test p P=0.003) were significantly increased in patients with tumors characterized by high βalt scores.

These striking patient data show that, despite different tissue origins and treatment regimens, a high βalt score is consistently associated with better outcome for cancer patients treated with genotoxic agents. This provides compelling evidence that the mechanisms by which TGFβ impacts alt-EJ repair have significant biological and clinical consequences, including robust associations with genomic alterations and response to cancer therapy.

Pan-cancer anti-correlation of TGFβ competency and alt-EJ genes The coordinated expression of alt-EJ genes in HNSC was unanticipated because these genes have not previously been identified as a network or pathway. To further evaluate this observation, consensus clustering of both gene sets was conducted across all TCGA solid cancers (n=10,848;). As expected, subsets of TGFβ target genes clustered together, which is likely due to the pleiotropic actions of TGFβ in both cancer cells and the tumor microenvironment. Remarkably, a block containing 27 of the alt-EJ signature genes indicates that they are highly co-regulated.

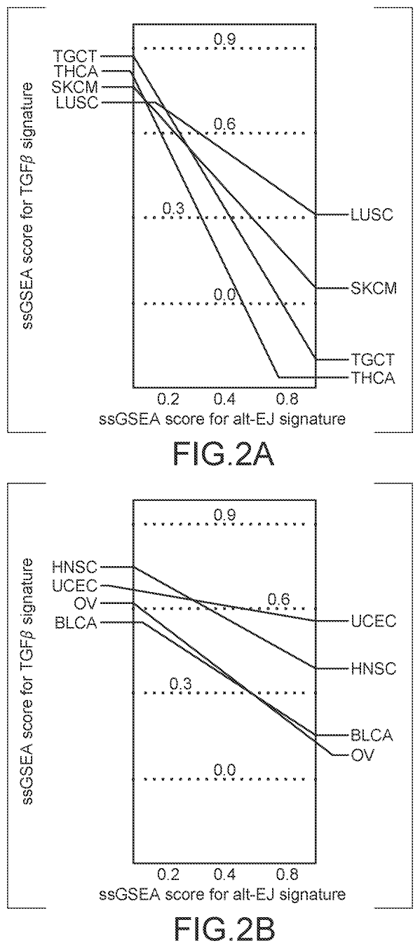

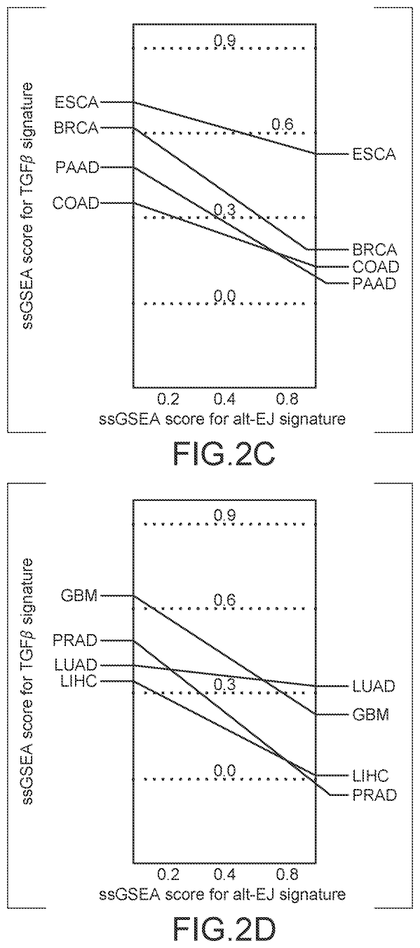

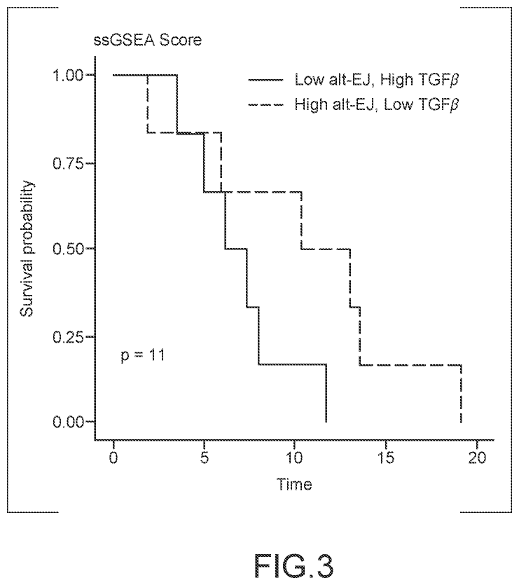

Among the 17 cancer types analyzed, 16 showed significant anti-correlation between the alt-EJ and TGFβ signatures (FIGS. 2A, 2B, 2C, and 2D). These data indicate that this relationship is broadly present in human cancer.

TGFβ signaling in the tumor microenvironment affects diverse responses within and between tumor, immune and stromal cells, any of which may contribute to the relationship between TGFβ and alt-EJ. To assess this, immune and stromal cell inference was used to test the association of these factors with TGFβ/alt-EJ signatures across different cancer types. There were no specific associations of the signatures with inferred immune and stromal cell contents, demonstrating that TGFβ suppression of an alt-EJ program is a cancer-cell autonomous feature.

To assess the cancer cell autonomy of this relationship further, the the two signatures were analyzed across multiple cancer cell lines. As observed in primary tumors, the overall negative correlation was strongly maintained (n=966, PCC=−0.35, p<0.00001) and negative correlations were observed in most cancer cell types, which included cell lines from GBM (n=35, PCC=−0.43, P<0.01), HNSC (n=42, PCC=−0.57, P<0.001), and LUSC (n=15, PCC=−0.68, P<0.001).

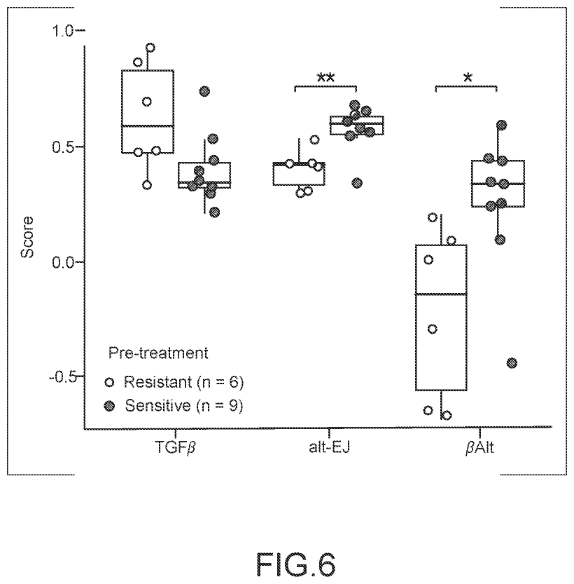

Pan-cancer TGFβ and alt-EJ signatures associate with specific microhomology indel mutation and survival after genotoxic therapy. Because the TGFβ and alt-EJ signatures showed a universal negative correlation across solid cancers, functional consequences were investigated. The alt-EJ process is inherently mutagenic because it uses sequence micro-homologies to facilitate DSB ligation. Hence, we predicted that the TGFβ and alt-EJ signature relationship would be associated with the frequency of specific genomic alterations across cancers. To evaluate this, signature scores in tumors were assessed for their association with the somatic frequencies of small insertions and deletions, and with silent non-coding mutations. The alt-EJ signature was positively correlated with higher frequencies of these mutation types in most cancers. The average distribution of the observed PCC for alt-EJ was significantly higher than 0 (t-test, P<0.00001). In contrast, the TGFβ signature was negatively correlated with these types of mutations in several cancer settings (PCC average <0, t-test, P=0.009). Consistent with these results, the frequency of distinct types of somatic structural variants, including chromosomal translocations, was also positively correlated with the alt-EJ signature and negatively with the TGFβ signature (FIG. 6).

Next was performed a comprehensive analysis of mutational signatures of cancer genomes in which it was focused on insertion-deletion (indel) signatures. Those designated ID6 and ID8 are characterized by >5-base pair deletions, but ID6 contains overlapping microhomology at deletion boundaries with a mode of 2 base pair. This signature pattern is consistent with end-joining by either NHEJ or alt-EJ. Notably, a similar pattern can be experimentally induced in cells in a Pol dependent manner. Samples were matched to TCGA analyses of TGFβ and alt-EJ expression signatures and the correlation for each gene set with ID pattern probabilities was analyzed. The resulting heatmap of PCC showed that the gene signatures are differentially associated with ID patterns. ID6 showed a striking positive correlation with the alt-EJ signature, whilst it was negatively correlated with TGFβ gene targets expression. ID10 and ID13 showed the opposite correlation with TGFβ and alt-EJ. The reciprocal correlation of alt-EJ and TGFβ with ID6 indicates that it is a genomic scar of Pol dependent alt-EJ, which further endorses their functional relationship.

Given the evidence that the biology represented in these signatures did not depend on cancer type, an OS pan-cancer analysis were performed for patients who were treated with RT (n=1737). The anti-correlation of signatures was comparable to all specimens and represented 17 malignancies. Patients with high βalt scores fared significantly better (p P=0.0001, hazard ratio=1.34, 95% CI 1.17-1.52) than those with low βalt scores. Because chemotherapy is not reported in detail in TCGA, patients were selected based on type and stage for whom SOC would include RT and/or genotoxic ChT (n=3577). The signature anti-correlation was comparable to all specimens and represented 17 malignancies. Patients with high βalt scores again showed better survival (P<0.0001; hazard ratio=1.29, 95% CI 1.2-1.38). Thus, survival duration in response to genotoxic therapy associates with implementation alt-EJ upon loss of TGFβ competency.

The results described herein demonstrate a novel reciprocal relationship between TGFβ signaling and alt-EJ-mediated DNA damage repair that has fundamental implications in cancer biology, progression and therapeutic vulnerability. Proteomic, gene expression, and functional evidence from HNSC and GBM cancer cells demonstrate that TGFβ signaling has extensive control over DNA damage responses. In contrast to the TGFβ-miR-182-BRCA1 axis that regulates HR, TGFβ inhibition increases the use of alt-EJ—and expression of key components in this process—in a miR-182-independent manner. The alt-EJ process is both inefficient and error-prone, which leads to more residual damage and cell death. Alt-EJ and TGFβ competency signatures are almost universally anti-correlated in solid cancers, reciprocally linked to recently reported indel signature ID6, and predict survival benefit from genotoxic therapies across all cancer patients studied.

The highly significant anti-correlation of TGFβ and alt-EJ signatures across multiple cancer types and cancer cell lines was unexpected, revealing a functional network phenotype. Notably, altEJ genes have not previously been shown to form a network, and they are not known to be TGFβ target genes.

Herein was identified a specific DNA repair deficit and means of assessing the same, including a newly defined βalt score. These advances will be useful for directing subjects to optimal treatments, exploitable within the current cancer therapy repertoire.

Gene expression analyses methods: Total RNA was extracted. Sample RNA (250 ng) was used for the NANOSTRING™ DNA Damage Repair Gene Expression panel, consisting of 180 DDR genes and 12 housekeeping genes for normalization, according to manufacturer's instructions. Hybridization efficiency and background signals were evaluated based on internal positive and negative control probes analyzed using manufacturer's software. Ratio data from 2-3 biological repeats were used for further analysis and graphs.

### Example 2. Glioblastoma Multiform (GMB) and TGF β/Alt-EJ Gene Signatures

In GBM, the analysis revealed an overwhelmingly significant negative correlation of the TGFβ and alt-EJ signatures using TCGA data from microarrays (n=442, PCC=−0.35, p<10⁻¹⁴) or RNAseq (n=131, PCC=−0.41, p<10⁻⁷). Furthermore, the low TGFβ/high alt-EJ profile in GBM is associated with significantly better progression-free survival (p<0.003) and overall survival (p<0.02) in patients receiving standard of care chemoradiotherapy. Furthermore RNA sequencing data from 12 GBM patients receiving olaparib and radiotherapy in the PARADIGM phase I trial shows a strong association between overall survival and expression of the low TGFβ/high alt-EJ gene signature.

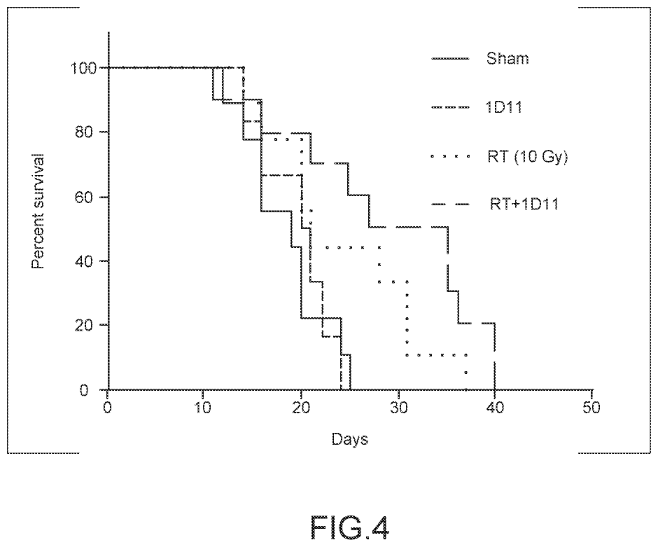

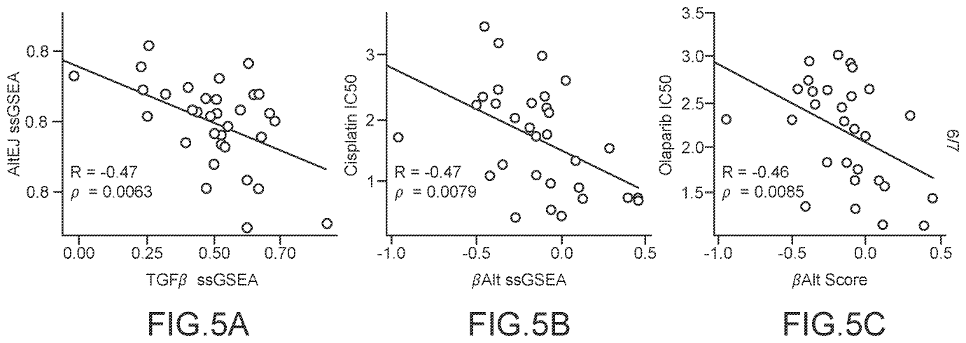

In animal work, GL261 and SB28 syngeneic intracranial murine GBM models were used. To test the effects of 1D11 antibody on radioresponse, mice bearing murine GBM SB28 intracranial tumors, confirmed by IVIS and contrast-enhanced CT scan, were randomized based on tumor BLI and irradiated with a single dose of 10 Gy, 6-10 days post inoculation. Fresolimomab (GC1008) is a humanized version of monoclonal antibody 1D11. 1D11 (10 mg/kg) was administered i.p. 24 hr before the first dose and every 3 days for the next 14 days. Little survival benefit was observed in mice treated with 1D11 alone (20.5 days median survival) compared to sham antibody-treated mice (19 days). While 10 Gy only modestly increased survival (21 days), addition of 1D11 to radiation significantly improved survival (31 days) (FIG. 4), an effect also seen with GL261 intracranial tumors. Thus, TGFβ inhibition in a GBM intracranial model produces radiosensitization.

Example 3. Functional validation of TGFβ signaling and alternative end-joining DNA repair signatures and their predictive utility in genotoxic cancer therapy. The significant association of PAU and patient survival was confirmed in independent ovarian cancer and HNSC datasets. To facilitate application to retrospective analysis of human specimens, a targeted approach was employed to analyze expression 200 genes associated with TGFβ and DDR using NanoString technology for direct counting of RNA transcripts without the need for amplification, as described in Geiss, et al., 2008. Direct multiplexed measurement of gene expression with color-coded probe pairs. Nature biotechnology 26, 317-325. The custom panel consists of 50 genes induced by chronic TGFβ, 36 genes necessary for execution of alt-EJ, and 12 housekeeping genes. Extracted RNA from 15 HNSC patient derived xenografts (PDX) and 22 primary HNSC specimens were used for evaluation.

Unsupervised hierarchical clustering of the HNSC tumors and their transcriptomic phenotype was conducted. As observed using RNAseq data from TCGA, most TGFβ genes or alt-EJ genes clustered together, but apart from each other. Most genes from the same signature are coordinately expressed together in the same module and are highly interconnected, thus indicating that their transcription is coregulated. Conversely, most TGFβ and altEJ genes are anticorrelated, evidencing that the counter relationship between TGFβ signaling and altEJ is also reproducible and concordant at the single gene scale. HNSC specimens were grouped into two major clades: one characterized by low TGFβ and high altEJ genes expression and the other showing the opposite pattern. As found previously, most HPV-positive samples gathered in the dendrogram arm with low TGFβ and high altEJ. The presence of HPV-negative tumors in that cluster denoted that mechanisms other than HPV status can lead to this phenotype, supporting conclusion that the reciprocal association between the gene sets is not simply a surrogate for HPV status. Notably, PDX and primary patient tumor specimens were distributed among both clades, suggesting that their TGFβ and altEJ transcriptional profiles were similar, as reported for proteomic profiles.

Short-term explants of the same PDX and primary tumor specimens to measure TGFβ pathway activity by frequency of cells exhibiting phosphorylated SMAD 2 (pSMAD2), a main downstream effector of TGFβ. DNA repair proficiency was measured 5 hours after irradiation by immunostaining of 53BP1, which forms foci at initial DSB sites but are resolved upon repair. Unrepaired DNA damage remain marked by 53BP1 REF. The percentage of pSMAD2 positive cells and the percentage of cells with 53BP1 foci 4 hr after irradiation were quantified in each specimen.

HNSC explants showed heterogenous frequencies of pSMAD2 and 53BP1 positive cells, whose frequencies were anti-correlated. The percentage of pSMAD2 and 53BP1 positive cells were negatively correlated across all specimens (Spearman's correlation coefficient (SCC)=−0.48, P<0.045), demonstrating that loss of TGFβ signaling is negatively correlated with ineffective DNA damage repair. Consistent with earlier work, HPV-positive samples n=3) had a low percentage of pSMAD2 positive cells and high levels of unrepaired DNA.

The TGFβ gene expression score was significantly correlated with the percentage of pSMAD2 positive cells (SCC=0.38, P=0.023), demonstrating that it is functionally indicative of TGFβ signaling status. Likewise, the altEJ gene expression score was positively correlated with the percentage of cells with residual 53BP1 foci after irradiation (SCC=0.51, P=0.023), consistent with the signature represents a functional biological process. As expected, TGFβ and altEJ scores were negatively correlated (PCC=0.4, P<0.013).

To further explore the value of the βalt_{w} score, evaluated outcomes in HNSC patients after excluding those whose primary curative treatment had been surgery (n=419) were evaluated. The weighted TGFβ and altEJ signatures were significantly anti-correlated (PCC=−0.35, P<10⁻¹⁶). Notably, patients with high βalt_{w} scores had significantly better OS than those with low βalt_{w} scores (log-rank test, P=0.017).

Next a small dataset consisting exclusively of HPV-negative oropharyngeal HNSC (n=97; GSE41613), was analyzed. Patients high βalt_{w} scores had significantly better OS than those with low βalt_{w} scores (P −0.0084). These data indicate that the predictive value of the relationship between TGFβ and altEJ is relevant beyond prognosis based on HPV status.

Ovarian cancer is sensitive to platinum-based chemotherapy and the current standard is carboplatin and paclitaxel in the first-line setting. Previous results using TCGA data was independently validated using data from previously untreated late stage II-III high grade OVCA patients who were treated with adjuvant platinum chemotherapy (GSE26712-OV, n=185). TGFβ patients whose disease was suboptimal debulking were used to evaluate βalt_{w} performance (n=95). The differential between patients with high vs low scores was significant (P=0.0051). Indeed, suboptimal debulked patients with high βalt_{w} fared as well as those optimally debulked patients, while those with low βalt_{w} had substantially poorer outcomes (P=0.0014).

All patents, patent applications, and publications cited in this specification are herein incorporated by reference to the same extent as if each independent patent application, or publication was specifically and individually indicated to be incorporated by reference. The disclosed embodiments are presented for purposes of illustration and not limitation. While the invention has been described with reference to the described embodiments thereof, it will be appreciated by those of skill in the art that modifications can be made to the structure and elements of the invention without departing from the spirit and scope of the invention as a whole.

What is claimed is:

1. A method of assessing DDR deficit phenotype in cancer cells of a subject;
wherein the DDR deficit phenotype comprises impaired TGFβ signaling and alt-EJ activation;
the method comprising the steps of:
obtaining a sample comprising cancer cells from the subject;
assessing TGFβ signaling competency in the cancer cells of the sample;
assessing alt-EJ activation in the cancer cells of the sample;
wherein, if impaired TGFβ signaling and alt-EJ activation is observed, the cancer cells of the subject are deemed to have the DDR deficit phenotype.

2. The method of claim 1 wherein
TGFβ signaling competency is assessed by measurement of one or more TGFβ-associated genes selected from the group consisting of:
ABCG1, AMIGO2, CA12, CCDC99, CCL20, CHRNA9, COL4A2, CTGF, DLC1, DNAJB9, DSC2, ENC1, ENC1, F3, FAP, FGF2, FGF2, FN1, HEY1, HMGA2, ID1, IGF2BP3, IGFBP3, JAG1, KLF4, LAMB3, LAMC2, LARP6, LIPG, MAFF, MMD, PDGFC, PLEK2, PLXNA2, POSTN, PSCD1, RICS, RNF24, RUNX1, SAMSN1, SERPINE1, SERPINE2, SH2D2A, SH2D4A, SLC20A1, SLC22A4, TGIF1, THBS1, TMEPAI, TNC, TNFRSF12A, and VCAN;
wherein low expression of the selected genes is indicative of impaired TGFβ signaling in the cancer cells.

3. The method of claim 2, wherein
the one or more TGFβ-associated genes comprises any of FAP, FN1, POSTN, SERPINE1, and THB S1.

4. The method of claim 3, wherein
the one or more TGFβ-associated genes comprises FAP, FN1, POSTN, SERPINE1, and THBS1.

5. The method of claim 1, wherein
alt-EJ activation is assessed by measurement of one or more Alt-EJ-associated genes selected from the group consisting of:
APE2, APEX1, ASF1A, CDKN2D, CIB1, DNA2, FAAP24, FANCM, GEN1, HARAS1, LIG1, LIG3, MEN1, MRE11A, MSH3, MSH6, MTH1, MTOR, NAPB2, NTHL1, PALB2, PARP1, PARP3, POLA1, POLM, POLQ, PRP19, RAD51D, RBBP8, RRM2, RUVBL2, SOD1, TIP60, UNG, WRN, and XRCC1 wherein high expression of the selected genes is indicative of Alt-EJ activation in the cancer cells.

6. The method of claim 5, wherein
the one or more alt-EJ-associated genes comprises any of any of GEN1, RRM2, DNA2, POLQ, and LIG1.

7. The method of claim 5, wherein
the one or more alt-EJ-associated genes comprises GEN1, RRM2, DNA2, POLQ, and LIG1.

8. The method of claim 1, wherein
TGFβ signaling competency and Alt-EJ activation are assessed by measurement of the expression of selected TGFβ-associated genes and alt-EJ-associated genes in cancer cells of the sample by an integrated score.

9. The method of claim 8, wherein
the integrated score is a β-alt score.
wherein an PAU score above a selected threshold indicative of the DDR deficit phenotype.

10. The method of claim 1, wherein
the sample comprises cancerous tissue, potentially cancerous tissue, or precancerous tissue.

11. The method of claim 1, wherein
the sample comprises a biopsy, tissue section, a paraffin-embedded tissue block, isolated cells, blood, serum, or cultured cells derived from an explant.

12. The method of claim 1, wherein
the cancer cells comprise cells of a carcinoma, sarcoma, or hematopoietic cancer.

13. The method of claim 12, wherein
the cancer is a carcinoma selected from the group consisting of bladder cancer, brain cancer, breast cancer, cervical cancer, colorectal cancer, endometrial cancer, esophageal cancers, gastric cancer, glioblastoma, glioma, head and neck cancer, lung cancer, melanoma, mesothelioma, nasopharyngeal cancer, ovarian cancer, pancreatic cancer, prostate cancer, renal cancer, testicular cancer, thyroid cancer, skin cancer, and uterine cancer.

14. The method of claim 12, wherein
the cancer is a sarcoma selected from the group consisting of undifferentiated pleomorphic sarcoma, epithelioid sarcoma, liposarcoma, and leiomyosarcoma.

15. The method of claim 12, wherein
the cancer is a hematopoietic cancer selected from the group consisting of leukemia, lymphoma, and myeloma.

16. A method of treating cancer in a subject in need of treatment therefor, comprising
assessing whether the cancer cells of the subject have the DDR deficit phenotype by the method of any of claims 1-15;
wherein, if the cancer cells of the subject are determined to have the DDR deficit phenotype, the subject is administered one or more treatments selected from the group consisting of a genotoxic therapy, PARP inhibition, or an immunotherapy.

17. The method of claim 16, wherein
the cancer cells comprise cells of a carcinoma, sarcoma, or hematopoietic cancer.

18. The method of claim 17, wherein
the cancer is a carcinoma selected from the group consisting of bladder cancer, brain cancer, breast cancer, cervical cancer, colorectal cancer, endometrial cancer, esophageal cancers, gastric cancer, glioblastoma, glioma, head and neck cancer, lung cancer, melanoma, mesothelioma, nasopharyngeal cancer, ovarian cancer, pancreatic cancer, prostate cancer, renal cancer, testicular cancer, thyroid cancer, skin cancer, and uterine cancer.

19. The method of claim 17, wherein
the cancer is a sarcoma selected from the group consisting of undifferentiated pleomorphic sarcoma, epithelioid sarcoma, liposarcoma, and leiomyosarcoma.

20. The method of claim 17, wherein
the cancer is a hematopoietic cancer selected from the group consisting of leukemia, lymphoma, and myeloma.

21. The method of claim 16, wherein
the one or more treatments comprises a genotoxic treatment.

22. The method of claim 21, wherein
the genotoxic comprises administration to the subject of a chemotherapeutic agent.

23. The method of claim 22, wherein
the chemotherapeutic agent is an agent comprising platinum.

24. The method of claim 21, wherein
the genotoxic treatment comprises the administration of a therapeutically effective amount of ionizing radiation to cancer cells of the subject.

25. The method of claim 24, wherein
the ionizing radiation is administered as an external beam treatment.

26. The method of claim 24, wherein
the ionizing radiation is administered as a radiopharmaceutical.

27. The method of claim 26, wherein
the radiopharmaceutical comprises a small molecule, peptide, antibody, nanoconstruct, or microsphere that preferentially targets cancer cells of the subject.

28. The method of claim 24, wherein
the ionizing radiation is administered as a brachytherapy implant.

29. The method of claim 16, wherein
the one or more treatments comprises administration to the subject of a therapeutically effective amount of a PARP inhibitor.

30. The method of claim 30, wherein
the PARP inhibitor is selected from the group consisting of olaparib, rucparib, niraparib, talazoparaib, veliparib, pamiparib, AG1436,10EP 9722, E7016, 3-aminobenzamide, and BGB-290.

31. The method of claim 16, wherein
the one or more treatments comprises an immunotherapy.

32. The method of claim 31, wherein
the immunotherapy comprises administration to the subject of a therapeutically effective amount of an agent selected from the group consisting of:
an immune checkpoint inhibitor; an inhibitor of CTLA-4; Ipilimumab, an inhibitor of PD-1, Nivolumab, Pembrolizumab, an inhibitor of PD-L1, Atezolizumab, Avelumab, Durvalumab, a cellular immunotherapy agent, dendritic cells that have been primed ex-vivo, chimeric antigen receptor T-cells, Tsagenlecleucel, axicabtagene ciloleucel, tumor-infiltrating lymphocytes primed ex-vivo, tumor lysate, a cytokine, interferon-alpha, interleukin-2, and GM-CSF.

33. A kit, comprising
a plurality of components which may be used in carrying out an assessment of TGFβ signaling competency and alt-EJ activation in cancer cells of a sample.

34. The kit of claim 33, comprising
a plurality of components for the quantification of expression of selected TGFβ-associated genes in cancer cells of the sample; and
a plurality of components for the quantification of the expression of selected alt-EJ-associated genes in the sample.

35. The kit of claim 34, wherein
the selected TGFβ-associated genes comprise one or more genes selected from the group consisting of:
ABCG1, AMIGO2, CA12, CCDC99, CCL20, CHRNA9, COL4A2, CTGF, DLC1, DNAJB9, DSC2, ENC1, ENC1, F3, FAP, FGF2, FGF2, FN1, HEY1, HMGA2, IGF2BP3, IGFBP3, JAG1, KLF4, LAMB3, LAMC2, LARP6, LIPG, MAFF, MMD, PDGFC, PLEK2, PLXNA2, POSTN, PSCD1, RICS, RNF24, RUNX1, SAMSN1, SERPINE1, SERPINE2, SH2D2A, SH2D4A, SLC20A1, SLC22A4, TGIF1, THBS1, TMEPAI, TNC, TNFRSF12A, and VCAN.

36. The kit of claim 35, wherein
the selected TGFβ-associated genes comprise one or more of FNI, FAP, POSTN, THBS1, and SERPINE1.

37. The kit of claim 35, wherein
the one or more selected TGFβ-associated genes comprises FNI, FAP, POSTN, THBS1, and SERPINE1.

38. The kit of claim 34, wherein
the Alt-EJ-associated genes comprise one or more genes selected from the group consisting of:
APE2, APEX1, ASF1A, CDKN2D, CIB1, DNA2, FAAP24, FANCM, GEN1, HARAS1, LIG1, LIG3, MEN1, MRE11A, MSH3, MSH6, MTH1, MTOR, NAPB2, NTHL1, PALB2, PARP1, PARP3, POLA1, POLM, POLQ, PRP19, RAD51D, RBBP8, RRM2, RUVBL2, SOD1, TIP60, UNG, WRN, and XRCC1.

39. The kit of claim 38, wherein
the one or more alt-EJ-associated genes comprises one or more of GEN1, RRM2, DNA2, LIG1, and POLQ.

40. The kit of claim 38, wherein
the one or more alt-EJ-associated genes comprises GEN1, RRM2, DNA2, LIG1, and POLQ.

41. The kit of any of claims 34-40, wherein
the kit comprises:
a plurality of probes; wherein each probe comprises an oligonucleotide sequence that will selectively hybridize with a transcript of or cDNA of selected TGFβ-associated genes; and
a plurality of probes wherein each probe comprises a nucleus acid sequence that will selectively hybridize with a mRNA transcript of or cDNA of selected alt-EJ-associated genes.

42. The kit of claim 41, wherein
the probes comprise barcoded constructs.

43. The kit of claim 41, wherein
the probes are immobilized on a substrate.

44. The kit of any of claims 34-40, wherein
the kit comprises:
a plurality of PCR primer pairs; wherein each primer pair will selectively amplify a transcript of or cDNA of selected TGFβ-associated genes; and
a plurality of primer pairs wherein each primer pair will selectively amplify a transcript of or cDNA of selected alt-EJ-associated genes.

45. An inhibitor of TGFβ signaling activity for use in a method of treating cancer in a subject, wherein
the method of treating cancer in a subject comprises:
administering to the subject a first treatment comprising administration to the subject of a therapeutically effective amount of an inhibitor of TGFβ signaling activity; and
administering to the subject one or more additional treatments selected from a genotoxic treatment; PARP inhibition; and an immunotherapy treatment.

46. The inhibitor of TGFβ signaling activity of claim 45, wherein
the cancer cells comprise cells of a carcinoma, sarcoma, or hematopoietic cancer.

47. The inhibitor of TGFβ signaling activity of claim 46, wherein
the cancer is a carcinoma selected from the group consisting of bladder cancer, brain cancer, breast cancer, cervical cancer, colorectal cancer, endometrial cancer, esophageal cancers, gastric cancer, glioblastoma, glioma, head and neck cancer, lung cancer, melanoma, mesothelioma, nasopharyngeal cancer, ovarian cancer, pancreatic cancer, prostate cancer, renal cancer, testicular cancer, thyroid cancer, skin cancer, and uterine cancer.

48. The inhibitor of TGFβ signaling activity of claim 46, wherein
the cancer is a sarcoma selected from the group consisting of undifferentiated pleomorphic sarcoma, epithelioid sarcoma, liposarcoma, and leiomyosarcoma.

49. The inhibitor of TGFβ signaling activity of claim 46, wherein
the cancer is a hematopoietic cancer selected from the group consisting of leukemia, lymphoma, and myeloma.

50. The inhibitor of TGFβ signaling activity of claim 45, wherein
the inhibitor of TGFβ signaling activity is selected from the group consisting of:
a neutralizing antibody, a ligand trap, a kinase inhibitor, an antisense composition, human monoclonal antibody 264RAD; fresolimumab; a human antibody neutralizing TGFβ1; LY3022859; an anti-TβRII monoclonal antibody that inhibits receptor-mediated TGFβ signaling activation; galunisertib; a TβRI kinase inhibitor; Belagenpumatucel-L; gemogenovatucel-T, trabedersen, XOMA089; SB-431542; SB-545344; SB-505124; LY2109761; LY364947; IN-1130; SD-208; R-268712; A-7701; A-83-01; GW788388; pirfenidone; fluorofenidonel; C11300; Sotatercept; and Luspatercept.

51. The inhibitor of TGFβ signaling activity of claim 45, wherein
the one or more additional treatments comprises a genotoxic treatment.

52. The inhibitor of TGFβ signaling activity of claim 51, wherein
the genotoxic comprises administration to the subject of a chemotherapeutic agent.

53. The inhibitor of TGFβ signaling activity of claim 45, wherein
the chemotherapeutic agent comprises platinum.

54. The inhibitor of TGFβ signaling activity of claim 51, wherein
the genotoxic treatment comprises the administration of a therapeutically effective amount of ionizing radiation to cancer cells of the subject.

55. The inhibitor of TGFβ signaling activity of claim 54, wherein
the ionizing radiation is administered as an external beam treatment.

56. The inhibitor of TGFβ signaling activity of claim 54, wherein
the ionizing radiation is administered as a radiopharmaceutical.

57. The inhibitor of TGFβ signaling activity of claim 56, wherein
the radiopharmaceutical comprises a small molecule, peptide, antibody, nanoconstruct, or microsphere that preferentially targets a radioactive composition to cancer cells of the subject.

58. The inhibitor of TGFβ signaling activity of claim 54, wherein
the ionizing radiation is administered as a brachytherapy implant.

59. The inhibitor of TGFβ signaling activity of claim 45, wherein
the one or more treatments comprises administration to the subject of a therapeutically effective amount of a PARP inhibitor.

60. The inhibitor of TGFβ signaling activity of claim 59, wherein
the PARP inhibitor is selected from the group consisting of olaparib, rucparib, niraparib, talazoparaib, veliparib, pamiparib, AG1436,10EP 9722, E7016, 3-aminobenzamide, and BGB-290.

61. The inhibitor of TGFβ signaling activity of claim 45, wherein
the one or more additional treatments comprises an immunotherapy treatment.

62. The inhibitor of TGFβ signaling activity of claim 61, wherein
the immunotherapy treatment comprises the administration to the subject of a therapeutically effective amount of an immunotherapy agent selected from the group consisting of:
an immune checkpoint inhibitor; an inhibitor of CTLA-4; Ipilimumab, an inhibitor of PD-1, Nivolumab, Pembrolizumab, an inhibitor of PD-L1, Atezolizumab, Avelumab, Durvalumab, a cellular immunotherapy agent, dendritic cells that have been primed ex-vivo, chimeric antigen receptor T-cells, Tsagenlecleucel, axicabtagene ciloleucel, tumor-infiltrating lymphocytes primed ex-vivo, tumor lysate, a cytokine, interferon-alpha, interleukin-2, and GM-CSF.
